(19) United States
(12) Patent Application Publication
(10) Pub. No.: US 2026/0021029 A1
(43) Pub. Date: Jan. 22, 2026

(54) HAIR REPAIR AND PROTECTION

(71) Applicant: OLAPLEX, INC., Santa Barbara, CA (US)

(72) Inventors: Maria CHAVEZ, Pequannock, NJ (US); Renuka RATHI, Parsippany, NJ (US)

(21) Appl. No.: 19/277,136

(22) Filed: Jul. 22, 2025

Related U.S. Application Data
(60) Provisional application No. 63/673,947, filed on Jul. 22, 2024.

(51) Int. Cl. A61K 8/73; A61K 8/34; A61K 8/365; A61K 8/368; A61K 8/37; A61K 8/41; A61K 8/44; A61K 8/49; A61K 8/60; A61K 8/92; A61K 8/9789; A61Q 5/00; A61Q 5/12

(52) U.S. Cl. CPC A61K 8/737; A61K 8/34; A61K 8/365; A61K 8/368; A61K 8/375; A61K 8/41; A61K 8/44; A61K 8/4913; A61K 8/4946; A61K 8/4973; A61K 8/604; A61K 8/733; A61K 8/735; A61K 8/736; A61K 8/922; A61K 8/9789; A61Q 5/002; A61Q 5/12

(57) ABSTRACT

Methods and compositions for treating hair, such as to reduce split ends, seal cracks in hair cuticles, reduces damage to hair, and/or reduce frizziness and/or hair fly-aways. The methods and compositions use a combination of hydroxypropyl guar hydroxypropyltrimonium chloride, Argania spinosa kernel oil, guar hydroxypropyltrimonium chloride, and algin. In some instances, the composition further contains bis-aminopropyl diglycol dimaleate. In some instances, the composition further contains a fructooligosaccharide and/or Beta vulgaris root extract. In some instances, the composition further contains arginine, aspartic acid, glycine, alanine, serine, valine, proline, threonine, isoleucine, histidine, and/or phenylalanine.

### CROSS-REFERENCE TO RELATED PATENT APPLICATIONS

This application claims the benefit of U.S. Provisional Patent Application No. 63/673,947, filed Jul. 22, 2024. The contents of the referenced patent application is incorporated into the present application by reference.

### FIELD OF THE INVENTION

This invention general relates to the field of personal care methods and products that include hair treatments. In one aspect, the invention includes a composition that may be applied to hair that includes a beneficial combination of hydroxypropyl guar hydroxypropyltrimonium chloride, guar hydroxypropyltrimonium chloride, Argania spinosa kernel oil, and algin. The composition can provide beneficial results to the hair (e.g., repairs split ends, repairs cracks in hair cuticles, protects against heat damage, and/or reduces hair frizziness or hair fly-aways).

### BACKGROUND OF THE INVENTION

Hair care and styling products can be useful to improve the appearance of hair. For instance, such products can create the appearance of more voluminous hair, more shiny hair, and/or can change the shape of hair (e.g., introducing curls or straightening hair). Some known methods to introduce these appearances in hair include heat treatment, color treatment, chemical treatment, and/or exposing hair to ultraviolet light or radiation. While these treatments can provide for a desired appearance, the treated hair oftentimes becomes damaged and can suffer from dryness, brittleness or reduced strength, increased rough surface texture, loss of color, loss of natural shape, decreased luster, and/or stiffness.

There have been attempts to mitigate damage to hair from hair treatment processes (e.g., introduction of curls, straightening hair, color change, etc.). In one example, compositions formulated as solutions, foams, gels, or emulsions can oftentimes include ingredients to help treat the damaged or processed hair. Unfortunately, these compositions can result in greasy hair, weighed down hair, and/or sticky or tacky hair. Ultimately, current hair treatment formulations used to mitigate damage to hair processing techniques may exacerbate the hair damage and/or have a negative effect on the tactile properties of the hair.

Still further, in processes where hair is treated to, for example, change the shape of hair (e.g., introducing curls or straightening hair), there can be a heat treatment step (e.g., curling irons, blow dryers, etc.). Such heat treatment step can damage the hair shaft and/or undermine previous treatment steps. Efforts to counteract these issues have largely not been met with existing hair care products and treatment processes.

### SUMMARY OF THE INVENTION

A solution has been identified to at least one or more of the aforementioned problems associated with hair treatment processes. In one aspect, the solution can include a composition that may be applied to hair or a method of treating hair with the composition where the composition includes a beneficial combination of hydroxypropyl guar hydroxypropyltrimonium chloride, guar hydroxypropyltrimonium chloride, Argania spinosa kernel oil, and/or algin. With wishing to be bound by theory, it is believed that the composition can provide beneficial results to the hair (e.g., seals split ends, seals cracks in hair cuticles, protects against heat damage, reduces hair frizziness, reduces hair fly-aways, increases the elongation at break of individual hair fibers, increases the tensile strength of the hair, repairs broken disulfide bonds, protects hair from damage when heated at temperature of up to 235° C. or greater, such as 100, 105, 110, 115, 120, 125, 130, 135, 140, 145, 150, 155, 160, 165, 170, 175, 180, 185, 190, 195, 200, 205, 210, 215, 220, 225, 230, or 235° C., and/or forms disulfide bonds from broken disulfide bonds of the hair when compared to hair that has not been subjected to the method). Still further, the composition can be applied to and remain in the hair without being rinsed from the hair (e.g., leave-in-conditioner or moisturizer), which can be beneficial in helping protect the hair from heat damage caused by, for example, curling irons, flattening irons, blow dryers, hair dryers. A benefit of this thermal protection is that the hair can initially be treated with, for example, a composition of the present invention, and then subsequently subjected to a heat treatment step (e.g., 150° C. to 235° C., preferably 200° C. to 235° C.). The initial application of the composition to hair can protect the hair shaft and/or the components in the composition from a subsequent heat treatment step, which can be beneficial in that the initial treatment step is not negatively affected by a subsequent heat treatment step. Ultimately, the compositions of the present invention can provide a dual benefit in (1) initially treating the hair (e.g., repairing split ends, reducing hair frizziness or hair fly aways, preparing the hair for being shaped, etc.) and (2) protecting the hair from heat treatment at temperatures up to 235° C.

In some instances, the composition contains a combination of hydroxypropyl guar hydroxypropyltrimonium chloride, guar hydroxypropyltrimonium chloride, Argania spinosa kernel oil, and algin. The composition may contain hydroxypropyl guar hydroxypropyltrimonium chloride, guar hydroxypropyltrimonium chloride, Argania spinosa kernel oil, and algin, and any other ingredient disclosed herein at a concentration of 0.00000001 to 99.99999999 wt. % based on the weight of the composition. In some instances, the composition contains hydroxypropyl guar hydroxypropyltrimonium chloride at 0.01 to 1.5 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, or 1.5 wt. %. In some instances, the composition contains guar hydroxypropyltrimonium chloride at 0.01 to 1 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains Argania spinosa kernel oil at 0.01 to 1 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains algin at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %.

In some instances, the composition further contains bis-aminopropyl diglycol dimaleate. In some instances, the composition contains bis-aminopropyl diglycol dimaleate at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains along with the bis-aminopropyl diglycol dimaleate or instead of the bis-aminopropyl diglycol dimaleate a compound of Formula I

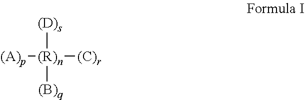

wherein:
A, B, C, and D are reactive moieties capable of reacting with the free thiol groups and contain one or more charges, and wherein each of A, B, C, and D independently comprises a vinyl sulfone, an acrylate group, a methacrylate group, a styrene group, an acryl amide group, a methacryl amide group, a maleate group, a fumarate group, and/or an itaconate group; and
R is a linker that contains two or more charges, wherein the charges are opposite to the charges on the reactive moieties, wherein n=1-10, and wherein R is not a polymer,
wherein each occurrence of p, q, r, and s is independently an integer from 0 to 25, and
wherein the sum of p+q+r+s is equal to or greater than 2. In some instances, the sum of the charges of the compound of Formula I is zero. In some instances, the reactive moieties are ionically bound to the linker.

In some instances, the composition contains the compound of Formula I at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %.

In some instances, the composition further contains fructooligosaccharide and/or Beta vulgaris root extract. In some instances, the composition contains fructooligosaccharide at 0.01 to 5 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 2, 2.5, 3, 3.5, 4, 4.5, or 5 wt. %. In some instances, the composition contains Beta vulgaris root extract at 0.01 to 3 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 2, 2.5, or 3 wt. %.

In some aspects, the composition further includes one or more of arginine, aspartic acid, glycine, alanine, serine, valine, proline, threonine, isoleucine, histidine, and/or phenylalanine. In some instances, the composition contains lactic acid at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains arginine at 0.001 to 2 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, or 2 wt. %. In some instances, the composition contains aspartic acid at 0.001 to 1.5 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, or 1.5 wt. %. In some instances, the composition contains glycine at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains alanine at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains serine at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains valine at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains proline at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains threonine at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains isoleucine at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains histidine at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains phenylalanine at 0.00001 to 1 wt. % by weight of the composition, such as equal to, more than, less than, a range of, or between 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %.

In some instances, the composition further comprises one or more of carrageenan, xanthan gum, chitosan, potassium lactate, lactic acid, sodium pyrrolidone carboxylic acid, pyrrolidone carboxylic acid, sodium lactate, caprylic/capric triglyceride, sorbitan oleate, coco-glucoside, phenoxyethanol, and/or sodium benzoate. In some instances, the composition contains carrageenan at 0.001 to 1 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains xanthan gum at 0.001 to 1 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains chitosan at 0.001 to 1 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains potassium lactate at 0.01 to 1 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains lactic acid at 0.001 to 1 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains sodium pyrrolidone carboxylic acid at 0.01 to 1.5 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, or 1.5 wt. %. In some instances, the composition contains pyrrolidone carboxylic acid at 0.001 to 1.5 wt. % by weight of the composition, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, or 1.5 wt. %. In some instances, the composition contains sodium lactate at 0.01 to 1.5 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, or 1.5 wt. %. In some instances, the composition contains caprylic/capric triglyceride at 0.01 to 2 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, or 2 wt. %. In some instances, the composition contains sorbitan oleate at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains coco-glucoside at 0.0001 to 1 wt. % by weight of the composition, such as equal to, more than, less than, a range of, or between 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %. In some instances, the composition contains phenoxyethanol at 0.01 to 5 wt. % by weight of the composition, such as equal to, more than, less than, a range of, or between 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 2, 2.5, 3, 3.5, 4, 4.5, or 5 wt. %. In some instances, the composition contains sodium benzoate at 0.000001 to 1 wt. % by weight of the composition, such as equal to, more than, less than, a range of, or between 0.000001, 0.000005, 0.00001, 0.00005, 0.0001, 0.0005, 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 wt. %.

In some instances, the composition further comprises or does not comprise one or more additional ingredient. The one or more additional ingredients may include water, preservatives, antioxidants, softeners, chelating agents, sunscreen agents, vitamins, dyes, hair coloring agents, pigments, fillers, proteins, amino acids, natural extracts such as plant extracts, moisturizing agents, humectants, fragrances, perfumes, oils such as essential oils or natural oils, emollients, surfactants, lubricants, butters, penetrants, thickeners, viscosity modifiers, polymers, resins, hair fixatives, film formers, surfactants, detergents, emulsifiers, opacifying agents, volatiles, propellants, foaming agents, antifoaming agents, liquid vehicles, carriers, salts, pH adjusting agents, neutralizing agents, buffers, pharmaceutically-active agents, hair conditioning agents, anti-static agents, anti-frizz agents, anti-dandruff agents, absorbents, and combinations thereof. The one or more additional ingredients may be any of the ingredients disclosed herein. In some aspects, the composition does not contain a silicone. In some aspects, the composition contains less than 0.5 wt. % of the silicone, such as equal to, less than, a range of, or between 0.5, 0.4, 0.3, 0.2, 0.1, 0.05, 0.01, 0.005, 0.001, 0.0005, 0.0001, 0.00001, or 0.00005 wt. %.

In some instances, the composition is a leave on hair care product such as a moisturizer, sunscreen, mask, styling gel, serum, or leave-in conditioner. In some instances, the composition is a rinse out hair care product such as a shampoo, conditioner, or soap.

In some instances, the composition has a viscosity of 10,000 to 300,000 cps, such as equal to, less than, a range of, or between 10,000, 20,000, 30,000, 40,000, 50,000, 60,000, 70,000, 80,000, 90,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, or 300,000 cps.

In some instances, the composition has a specific gravity of 0.8 to 1.5, such as equal to, less than, a range of, or between 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, or 1.5.

In some instances, the composition has a pH of 3.5 to 8, such as equal to, less than, a range of, or between 3.5, 4, 4.5, 5, 5.5, 6, 6.5, 7, 7.5, or 8.

In certain embodiments, the composition is comprised in a container. The container can be a bottle, dispenser, or package. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

In some aspects, methods of treating hair are disclosed herein. The methods may include applying the composition to hair. The method may include applying additional compositions to the hair, such as a composition comprising any one or more of the ingredients disclosed herein. The method may include applying these compositions separately or together to the hair. The method may include applying these compositions in any order to the hair. The method may include applying these compositions to hair without rinsing any one of or a combination of the compositions out of the hair.

In some instances, the method repairs split ends, such as 80% of split ends, such as equal to, more than, less than, a range of, or between 80, 81, 82, 83, 84, 85, 86, 87, 88, 89, 90, 91, 92, 93, 94, 95, 96, 97, or 98%, repairs cracks in hair cuticles, protects against heat damage at a temperature of up to 235° C. or greater, such as 100, 105, 110, 115, 120, 125, 130, 135, 140, 145, 150, 155, 160, 165, 170, 175, 180, 185, 190, 195, 200, 205, 210, 215, 220, 225, 230, or 235° C., reduces hair frizziness, reduces hair fly-aways, increases the elongation at break of individual hair fibers, increases the tensile strength of the hair, reduces the loss of disulfide bonds in the hair, and/or forms at least two disulfide bonds when compared to hair that has not been subjected to the method. In some aspects, the method forms at least two disulfide bonds from broken disulfide bonds of the hair with the bis-aminopropyl diglycol dimaleate and/or with the compound of Formula I.

In some instances, the composition stays on the hair without being rinsed off for at least 2 hours, such as equal to, more than, less than, a range of, or between 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours.

The method can be applied to any type of hair, such as straight, wavey, curly, crimpy, black, white, silver, grey, brown, auburn, red, blond, dyed, color treated, permed, straitened, and/or heat treated hair. In some instances, the hair is straight, curly, wavey, or crimpy hair that is naturally straight, curly, wavey, or crimpy. In some instances, the hair is wet or dry hair. In some instances, the method repairs split ends, such as 80% of split ends, such as equal to, more than, less than, a range of, or between 80, 81, 82, 83, 84, 85, 86, 87, 88, 89, 90, 91, 92, 93, 94, 95, 96, 97, or 98%, repairs cracks in hair cuticles, protects against heat damage, reduces hair frizziness, reduces hair fly-aways, increases the elongation at break of individual hair fibers, increases the tensile strength of the hair, reduces the loss of disulfide bonds in the hair at a temperature of up to 235° C. or greater, such as 100, 105, 110, 115, 120, 125, 130, 135, 140, 145, 150, 155, 160, 165, 170, 175, 180, 185, 190, 195, 200, 205, 210, 215, 220, 225, 230, or 235° C., and/or the forms at least two disulfide bonds from broken disulfide bonds of the hair with the bis-aminopropyl diglycol dimaleate and/or with the compound of Formula I when compared to hair that has not been subjected to the method for at least 18 hours, such as equal to, more than, less than, a range of, or between 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or 3, 4, 5, 6, 7, 8, 9, 10, 11, 12, 13, 14, 15, 16, 17, 18, 19, 20, or 21 days.

The method may further include styling the hair after application of the composition. The method may improve the retention of the styling of the hair. In some instances, the method improves the retention of the styling of the hair for at least 18 hours, such as equal to, more than, less than, a range of, or between 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or 3, 4, 5, 6, 7, 8, 9, 10, 11, 12, 13, 14, 15, 16, 17, 18, 19, 20, or 21 days. The method may include styling the hair within 3 hours of applying the composition to the hair, such as equal to, more than, less than, a range of, or between 5 minutes, 10 minutes, 15 minutes, 20 minutes, 25 minutes, 30 minutes, 35 minutes, 40 minutes, 45 minutes, 50 minutes, 55 minutes, 1 hour, 1 hour and 5 minutes, 1 hour and 10 minutes, 1 hour and 15 minutes, 1 hour and 20 minutes, 1 hour and 25 minutes, 1 hour and 30 minutes, 1 hour and 35 minutes, 1 hour and 40 minutes, 1 hour and 45 minutes, 1 hour and 50 minutes, or 1 hour and 55 minutes, 2 hours, 2 hours and 5 minutes, 2 hours and 10 minutes, 2 hours and 15 minutes, 2 hours and 20 minutes, 2 hours and 25 minutes, 2 hours and 30 minutes, 2 hours and 35 minutes, 2 hours and 40 minutes, 2 hours and 45 minutes, 2 hours and 50 minutes, 2 hours and 55 minutes, or 3 hours.

In some instances, the method does not change the color of the hair. In some instances, the method does not change the color of the hair once the hair is dried as compared to the hair before the method, or compared to the hair not subject to the method. The color of the hair and/or change in color of the hair may be determined by unaided human vision. The color of the hair that is not changed may be the natural color of the hair, the dyed color of the hair, the color treated color of the hair, and/or the bleached color of the hair.

In some aspects, disclosed herein is a kit that contains the composition. In some instances, the kit includes written instructions to perform any one of the methods disclosed herein. Also disclosed are the following aspects 1 to 20 of the present invention.

Aspect 1 is a method of treating a person's hair, the method comprising applying to the person's hair a composition comprising: (a) hydroxypropyl guar hydroxypropyltrimonium chloride; (b) Argania spinosa kernel oil; (c) guar hydroxypropyltrimonium chloride; and (d) algin, wherein application of the composition to the hair (1) seals at least 80% of split ends in the hair, (2) seals cracks in hair cuticles, (3) protects the hair from damage from heat up to 205° C., (4) reduces hair frizziness, and/or (5) reduces hair fly-aways.

Aspect 2 is the method of Aspect 1, wherein the hair comprises split ends and wherein application of the composition to the hair seals greater than 90% of split ends and/or greater than 95% of split ends of the hair.

Aspect 3 is the method of any one of Aspects 1 to 2, wherein the hair comprises cracks in the hair cuticles and wherein application of the composition to the hair seals at least a portion of the cracks in the hair cuticles.

Aspect 4 is the method of any one of Aspects 1 to 3, wherein application of the composition to the hair reduces hair frizziness and/or reduces hair fly-aways.

Aspect 5 is the method of any one of Aspects 1 to 4, further comprising heat treating the hair and wherein application of the composition to the hair at least partially protects the hair from damage from heat up to 205° C.

Aspect 6 is the method of any one of Aspects 1 to 5, wherein application of the composition to the hair increases the elongation at break of individual hair fibers in the hair.

Aspect 7 is the method of any one of Aspects 1 to 6, wherein application of the composition to the hair increases the tensile strength of the hair.

Aspect 8 is the method of any one of Aspects 1 to 7, wherein the composition is applied to wet hair.

Aspect 9 is the method of any one of Aspects 1 to 8, wherein the composition is not rinsed from the hair within 18 hours.

Aspect 10 is the method of any one of Aspects 1 to 9, wherein the composition includes less than 0.5 wt. %, preferably less than 0.1 wt. %, or more preferably 0 wt. % of a silicone.

Aspect 11 is the method of any one of Aspects 1 to 10, wherein the composition has: a viscosity of 50,000-150,000 cps; a specific gravity of 0.96 to 0.99; and a pH of 4.0 to 5.0.

Aspect 12 is the method of any one of Aspects 1 to 11, wherein the composition comprises, by weight of the composition: 0.25 to 0.75 wt. % of hydroxypropyl guar hydroxypropyltrimonium chloride; 0.5 to 0.5 wt. % Argania spinosa kernel oil; 0.1 to 0.5 wt. % guar hydroxypropyltrimonium chloride; and 0.001 to 0.2 wt. % algin.

Aspect 13 is the method of any one of Aspects 1 to 12, wherein the composition further comprises bis-aminopropyl diglycol dimaleate.

Aspect 14 is the method of Aspect 13, wherein the composition comprises 0.001 to 0.2 wt. % bis-aminopropyl diglycol dimaleate by weight of the composition.

Aspect 15 is the method of any one of Aspects 13 to 14, wherein the bis-aminopropyl diglycol dimaleate forms at least two disulfide bonds with one or more broken disulfide bond of the hair.

Aspect 16 is the method of any one of Aspects 1 to 15, wherein the composition further comprises: a fructooligosaccharide; Beta vulgaris root extract; arginine; aspartic acid; glycine; alanine; serine; valine; proline; threonine; isoleucine; histidine; and/or phenylalanine.

Aspect 17 is the method of Aspect 16, wherein the composition comprises, by weight of the composition: 0.1 to 2 wt. % of the fructooligosaccharide; 0.1 to 1.5 wt. % of Beta vulgaris root extract; 0.01 to 1 wt. % of arginine; 0.01 to 0.5 wt. % of aspartic acid; 0.001 to 0.1 wt. % of glycine; 0.001 to 0.1 wt. % of alanine; 0.001 to 0.1 wt. % of serine; 0.001 to 0.1 wt. % of valine; 0.0001 to 0.1 wt. % of proline; 0.0001 to 0.1 wt. % of threonine; 0.0001 to 0.1 wt. % of isoleucine; 0.0001 to 0.1 wt. % of histidine; and/or 0.0001 to 0.1 wt. % of phenylalanine.

Aspect 18 is the method of any one of Aspects 1 or 17, wherein the composition further comprises carrageenan, xanthan gum, chitosan, sodium benzoate, caprylic/capric triglyceride, sorbitan oleate, coco-glucoside, potassium lactate, lactic acid, sodium pyrrolidone carboxylic acid, pyrrolidone carboxylic acid, sodium lactate, and/or phenoxyethanol.

Aspect 19 is the method of Aspect 18, wherein the composition comprises, by weight of the composition: 0.1 to 2 wt. % phenoxyethanol; 0.1 to 1 wt. % of caprylic/capric triglyceride; 0.1 to 1 wt. % of sodium lactate; 0.01 to 1 wt. % of potassium lactate; 0.01 to 1 wt. % of sodium pyrrolidone carboxylic acid; 0.01 to 0.5 wt. % of pyrrolidone carboxylic acid; 0.001 to 0.5 wt. % of lactic acid; 0.001 to 0.1 wt. % carrageenan; 0.001 to 0.1 wt. % xanthan gum; 0.001 to 0.1 wt. % chitosan; 0.001 to 0.2 wt. % sorbitan oleate; 0.001 to 0.2 wt. % coco-glucoside; 0.00001 to 0.01 wt. % sodium benzoate.

Aspect 20 is the method of any one of Aspects 1 to 19, wherein the composition is a leave in conditioner.

The terms “a” and “an” are defined as one or more unless this disclosure explicitly requires otherwise. The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent. The terms “moiety”, “functional group”, “component”, and “compound” may be used interchangeably herein.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method that “comprises,” “has,” “includes” or “contains” one or more steps or elements possesses those one or more steps or elements, but is not limited to possessing only those one or more steps or elements. Likewise, an element of a system or composition that “comprises,” “has,” “includes” or “contains” one or more features possesses those one or more features, but is not limited to possessing only those one or more features.

Furthermore, a structure or composition that is configured in a certain way is configured in at least that way, but may also be configured in ways that are not listed. Metric units may be derived from the English units provided by applying a conversion and rounding to the nearest millimeter.

The feature or features of one embodiment may be applied to other embodiments, even though not described or illustrated, unless expressly prohibited by this disclosure or the nature of the embodiments. In any of the claims, the term “consisting of” or “consisting essentially of” can be substituted for any of the open-ended linking verbs recited above, in order to change the scope of a given claim from what it would otherwise be using the open-ended linking verb.

It is also contemplated that the compositions disclosed throughout this specification can be used as a leave-on or rinse-off composition. By way of example, a leave-on composition can be one that is topically applied to hair and remains on the hair for a period of time (e.g., at least 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the hair and then removed or rinsed from the hair (e.g., with water) within a period of time such as less than 5, 4, 3, 2, or 1 minute. An example of a rinse off composition can be a shampoo, conditioner, or soap. An example of a leave-on composition can be a moisturizer, sunscreen, mask, styling gel, serum, leave-in conditioner.

It is contemplated that any embodiment discussed in this specification can be implemented with respect to any method or composition of the invention, and vice versa. Furthermore, compositions of the invention can be used to achieve methods of the invention.

In one embodiment, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin and/or hair (e.g., compositions that are not too watery or greasy, compositions that have a silky texture, compositions that are non-tacky or sticky, etc.). Pharmaceutically or cosmetically elegant can also relate to the creaminess, weight, or lubricity properties of the composition or to the moisture retaining properties of the composition.

Also contemplated is a product comprising a composition of the present invention. In non-limiting aspects, the product can be a hair care product. The hair care product can be those described in other sections of this specification or those known to a person of skill in the art. Non-limiting examples of products include a hair moisturizer, a hair cream, a hair lotion, a hair softener, a hair gel, a hair wash, a hair cleanser, a hair sunscreen, a hair color-protecting composition, a hair mask, a hair perfume, a hair cologne, a shampoo, a conditioner, etc.

The terms “wt. %”, “% by weight”, “vol. %”, “% by volume”, “mol. %”, or “% by mol.” refers to a weight percentage of a component, a volume percentage of a component, or molar percentage of a component, respectively, based on the total weight, the total volume of material, or total moles, which includes the component. In a non-limiting example, 10 grams of component in 100 grams of the material is 10 wt. % of component.

A hair care composition as used herein may refer to a product that can be used to clean, treat or style hair. Examples of hair care compositions include, but are not limited to shampoos, conditioners, styling gels, aerosol styling sprays, non-aerosol styling sprays, aerosol styling mousses, styling gels, styling pomades, leave-in conditioning sprays, and thermal protection sprays.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is the ability to treat hair ((1) seals split ends, (2) seals cracks in hair cuticles, (3) protects against heat damage, (4) reduces hair frizziness, (5) reduces hair fly-aways, (6) increases the elongation at break of individual hair fibers, (7) increases the tensile strength of the hair, (8) reduces the loss of disulfide bonds in the hair at a temperature of up to 235° C., and/or (9) forms at least two disulfide bonds from broken disulfide bonds of the hair with the bis-aminopropyl diglycol dimaleate and/or with the compound of Formula I when compared to hair that has not been subjected to the method).

The term “hair” refers to one or more than one strand of hair. Hair also refers to virgin hair or processed hair, for example hair that has been exposed to hair waving, color treatment, and/or hair straightening formulations.

“Shampoo”, as used herein, generally refers to a liquid or semi-solid formulation applied to the hair that contains detergent or soap for washing the hair.

“Conditioner”, as used herein, generally refers to a formulation (e.g., liquid, cream, lotion, gel, semi-solid) applied to the hair to soften the hair, smooth the hair, and/or change the sheen of the hair.

“Analog” and “Derivative” are used herein interchangeably and refer to a compound that possesses the same core as the parent compound, but differs from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C1-C30 for straight chain, C3-C30 for branched chain), more preferably 20 or fewer carbon atoms, more preferably 12 or fewer carbon atoms, and most preferably 8 or fewer carbon atoms. In some embodiments, the chain has 1-6 carbons. Likewise, preferred cycloalkyls have from 3-10 carbon atoms in their ring structure, and more preferably have 5, 6 or 7 carbons in the ring structure. The ranges provided above are inclusive of all values between the minimum value and the maximum value.

The term “alkyl” includes both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

The alkyl groups may also contain one or more heteroatoms within the carbon backbone. Examples include oxygen, nitrogen, sulfur, and combinations thereof. In certain embodiments, the alkyl group contains between one and four heteroatoms.

“Alkenyl” and “Alkynyl”, as used herein, refer to unsaturated aliphatic groups containing one or more double or triple bonds analogous in length (e.g., C2-C30) and possible substitution to the alkyl groups described above.

“Aryl”, as used herein, refers to 5-, 6- and 7-membered aromatic rings. The ring may be a carbocyclic, heterocyclic, fused carbocyclic, fused heterocyclic, bicarbocyclic, or biheterocyclic ring system, optionally substituted as described above for alkyl. Broadly defined, “Ar”, as used herein, includes 5-, 6- and 7-membered single-ring aromatic groups that may include from zero to four heteroatoms. Examples include, but are not limited to, benzene, pyrrole, furan, thiophene, imidazole, oxazole, thiazole, triazole, pyrazole, pyridine, pyrazine, pyridazine and pyrimidine. Those aryl groups having heteroatoms in the ring structure may also be referred to as “heteroaryl”, “aryl heterocycles”, or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, perfluoroalkyl, and cyano. The term “Ar” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocycles, or both rings are aromatic.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, containing carbon and one to four heteroatoms each selected from non-peroxide oxygen, sulfur, and N(Y) wherein Y is absent or is H, O, (C1-4) alkyl, phenyl or benzyl, and optionally containing one or more double or triple bonds, and optionally substituted with one or more substituents. The term “heterocycle” also encompasses substituted and unsubstituted heteroaryl rings. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuranyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl.

“Heteroaryl”, as used herein, refers to a monocyclic aromatic ring containing five or six ring atoms containing carbon and 1, 2, 3, or 4 heteroatoms each selected from non-peroxide oxygen, sulfur, and N(Y) where Y is absent or is H, O, (C1-C8) alkyl, phenyl or benzyl. Non-limiting examples of heteroaryl groups include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), quinolyl (or its N-oxide) and the like. The term “heteroaryl” can include radicals of an ortho-fused bicyclic heterocycle of about eight to ten ring atoms derived therefrom, particularly a benzyl-derivative or one derived by fusing a propylene, trimethylene, or tetramethylene diradical thereto. Examples of heteroaryl include, but are not limited to, furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl (or its N-oxide), thienyl, pyrimidinyl (or its N-oxide), indolyl, isoquinolyl (or its N-oxide), quinolyl (or its N-oxide), and the like.

“Halogen”, as used herein, refers to fluorine, chlorine, bromine, or iodine.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C3-C20 cyclic, substituted C3-C20 cyclic, heterocyclic, substituted heterocyclic, amino acid, peptide, and polypeptide groups.

Heteroatoms, such as nitrogen, may have hydrogen substituents and/or any permissible substituents of organic compounds described herein that satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

The term “linker”, as used herein, refers to one or more polyfunctional, e.g. bifunctional molecules, trifunctional molecules, tetrafunctional molecules, etc., which can be used to ionically bound the two or more moieties and which do not interfere with the reactive properties of the moieties. The moieties may be attached to any part of the linker. Linkers can be a single atom, such as a heteroatom (e.g., O or S), a group of atoms, such as a functional group (e.g., amine, —C(═O)—, —CH2-), or multiple groups of atoms, such as an alkylene chain. Suitable linkers include but are not limited to oxygen, sulfur, carbon, boron, nitrogen, alkoxy, alkyl, alkenyl, cycloalkyl, cycloalkenyl, aryl, heterocycloalkyl, heteroaryl, ether, amine, and a polymer. The linker is optionally independently substituted with one or more substituents including hydrogen, halogen, cyano, alkoxy, alkyl, alkenyl, cycloalkyl, cycloalkenyl, aryl, heterocycloalkyl, heteroaryl, amine, hydroxy, formyl, acyl, carboxylic acid (—COOH), —C(O)R1, —C(O)OR1, carboxylate (—COO—), primary amide (e.g., —CONH2), secondary amide (e.g., —CONHR1), —C(O)NR1R2, —NR1R2, —NR1S(O)2R2, —NR1C(O)R2, —S(O)2R2, —SR1, and —S(O)2NR1R2, sulfinyl group (e.g., —SOR1), and sulfonyl group (e.g., —SOOR1); wherein R1 and R2 may each independently be hydrogen, alkyl, alkenyl, alkynyl, cycloalkyl, aryl, heterocycloalkyl and heteroaryl; wherein each of R1 and R2 is optionally independently substituted with one or more substituents selected from the group consisting of halogen, hydroxyl, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxyl, cycloalkyl optionally substituted with hydroxyl, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl. In some embodiments, the linker may be an alkoxy, ether, alkyl, alkenyl, cycloalkyl, cycloalkenyl, aryl, heterocycloalkyl, heteroaryl, amine, or a polymer. In some embodiments, the linker is not a polymer.

“Reactive moieties” are capable of reacting with a thiol group in the hair or on the skin to form a stable covalent bond. The reactive moiety is typically an electrophilic moiety capable of forming a salt with the linker. Alternately, the reactive moiety can be a free radical forming moiety. The reaction between the reactive moiety and the thiol groups may be initiated at room temperature and pressure when the reactive moiety contacts a thiol group in the hair or on the skin. In some embodiments, the reaction may require an initiator, such as heat, catalyst, basic conditions, or a free radical initiator. The rate of reaction between the reactive moiety and the thiol may be increased by changes in temperature, pH, and/or addition of one or more excipients, such as a catalyst; however, this is generally not required. In some embodiments, the reactive moieties are capable of undergoing a conjugate additional reaction. The reactive moieties can independently be or contain a Michael acceptor, a succinimidyl-containing group, a maleimido-containing group, azlactone, a benzoxazinone derivative, vinyl sulfone, vinyl sulfoximine, vinyl sulfonate, vinyl phosphonate, benzoxazinone, isocyanate, epoxide, an electrophilic moiety containing a leaving group, an electrophilic thiol acceptor, acrylic or acrylate group, a methacrylic or methacrylate group, a styrene group, an acryl amide group, a methacryl amide group, a maleate group, a fumarate group, an itaconate group, a vinyl ether group, an allyl ether group, an allyl ester group, a vinyl ester group, a sulfonate group, a phosphonate group, a sulfoxide group, a sulfonamide group, a sulfinimide group, a sulfinamide group, a sulfonimidate group, or a sulfonimidamide group. Examples of suitable reactive moieties and compounds of Formula I can be found in United States Patent Application Publication No. 2023/0201632A1, incorporated herein in its entirety.

“Soluble” in a solvent, such as water, as used herein, generally means at least 10, 50, 100, 125, 150, 200, 225, or 250 g is soluble in 1 L of the solvent at 25° C.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B—Example of Rating=1, No Split End Closure. The split end of each fiber is imaged using a 25× stereo microscope. A) shows before treatment and B) shows after treatment.

FIG. 2A and FIG. 2B—Example of Rating=2, MinimalDegree of Partial Closure (≤30% reduction in angle). The split end of each fiber is imaged using a 25× stereo microscope. A) shows before treatment and B) shows after treatment.

FIG. 3A and FIG. 3B—Example of Rating=3, ModerateDegree of Partial Closure (30-70% reduction in angle). The split end of each fiber is imaged using a 25× stereo microscope. A) shows before treatment and B) shows after treatment.

FIG. 4A and FIG. 4B—Example of Rating=4, ReasonableDegree of Partial Closure (≥70% reduction in angle). The split end of each fiber is imaged using a 25× stereo microscope. A) shows before treatment and B) shows after treatment.

FIG. 5A and FIG. 5B—Example of Rating=5, Complete Split End Closure. The split end of each fiber is imaged using a 25× stereo microscope. A) shows before treatment and B) shows after treatment.

FIG. 6A, FIG. 6B, and FIG. 6C—Example Scanning Electron Microscopy (SEM) images of Control for European hair at 600× magnification (A) and 2000× magnification (B) and (C).

FIG. 7A, FIG. 7B, and FIG. 7C—Example Scanning Electron Microscopy (SEM) images of second sample of Control for European hair at 600× magnification (A) and 2000× magnification (B) and (C).

FIG. 8A, FIG. 8B, and FIG. 8C—Example Scanning Electron Microscopy (SEM) images of third sample of Control for European hair at 600× magnification (A) and 2000× magnification (B) and (C).

FIG. 9A, FIG. 9B, and FIG. 9C—Example Scanning Electron Microscopy (SEM) images of 1× Method Treated European hair at 600× magnification (A) and 2000× magnification (B) and (C).

FIG. 10A, FIG. 10B, and FIG. 10C—Example Scanning Electron Microscopy (SEM) images of second sample of 1× Method Treated European hair at 600× magnification (A) and 2000× magnification (B) and (C).

FIG. 11A, FIG. 11B, and FIG. 11C—Example Scanning Electron Microscopy (SEM) images of third sample of 1× Method Treated European hair at 600× magnification (A) and 2000× magnification (B) and (C).

FIG. 12A, FIG. 12B, and FIG. 12C—Example Scanning Electron Microscopy (SEM) images of 3× Method Treated European hair at 600× magnification (A) and 2000× magnification (B) and (C).

FIG. 13A, FIG. 13B, and FIG. 13C—Example Scanning Electron Microscopy (SEM) images of second sample of 3× Method Treated European hair at 600× magnification (A) and 2000× magnification (B) and (C).

FIG. 14A, FIG. 14B, and FIG. 14C—Example Scanning Electron Microscopy (SEM) images of third sample of 3× Method Treated European hair at 600× magnification (A) and 2000× magnification (B) and (C).

FIG. 15A, FIG. 15B, and FIG. 15C—Example Scanning Electron Microscopy (SEM) images of Control for Curly hair at 600× magnification (A) and 2000× magnification (B) and (C).

FIG. 16A, FIG. 16B, and FIG. 16C—Example Scanning Electron Microscopy (SEM) images of second sample of Control for Curly hair at 600× magnification (A) and 2000× magnification (B) and (C).

FIG. 17A, FIG. 17B, and FIG. 17C—Example Scanning Electron Microscopy (SEM) images of third sample of Control for Curly hair at 600× magnification (A) and 2000× magnification (B) and (C).

FIG. 18A, FIG. 18B, and FIG. 18C—Example Scanning Electron Microscopy (SEM) images of 1× Method Treated Curly hair at 600× magnification (A) and 2000× magnification (B) and (C).

FIG. 19A, FIG. 19B, and FIG. 19C—Example Scanning Electron Microscopy (SEM) images of second sample of 1× Method Treated Curly hair at 600× magnification (A) and 2000× magnification (B) and (C).

FIG. 20A, FIG. 20B, and FIG. 20C—Example Scanning Electron Microscopy (SEM) images of third sample of 1× Method Treated Curly hair at 600× magnification (A) and 2000× magnification (B) and (C).

FIG. 21A, FIG. 21B, and FIG. 21C—Example Scanning Electron Microscopy (SEM) images of 3× Method Treated Curly hair at 600× magnification (A) and 2000× magnification (B) and (C).

FIG. 22A, FIG. 22B, and FIG. 22C—Example Scanning Electron Microscopy (SEM) images of second sample of 3× Method Treated Curly hair at 600× magnification (A) and 2000× magnification (B) and (C).

FIG. 23A, FIG. 23B, and FIG. 23C—Example Scanning Electron Microscopy (SEM) images of third sample of 3× Method Treated Curly hair at 600× magnification (A) and 2000× magnification (B) and (C).

FIG. 24—Image of tresses after wet combining. The order of tresses are from left to right, Control group, HGHC Treated group, Method Treated group, and Commercial Treated group.

FIG. 25—Image of tresses after dry combining. The order of tresses are from left to right, Control group, HGHC Treated group, Method Treated group, and Commercial Treated group.

FIG. 26—Image of tresses after dry combining further magnified and to show the frizziness of the tips. The order of tresses are from left to right, Control group, HGHC Treated group, Method Treated group, and Commercial Treated group.

### DETAILED DESCRIPTION

As noted above, methods and compositions for treating hair to repair split ends, repair cracks in hair cuticles, protect against heat damage, reduce hair frizziness or hair fly-aways, increase the elongation at break of individual hair fibers, increase the tensile strength of the hair, reduce damage to the hair at a temperature of up to 235° C., and/or forms at least two disulfide bonds from broken disulfide bonds of the hair with the bis-aminopropyl diglycol dimaleate and/or with the compound of Formula I are disclosed herein. The methods and compositions use a beneficial combination of two or more of hydroxypropyl guar hydroxypropyltrimonium chloride, guar hydroxypropyltrimonium chloride, Argania spinosa kernel oil, and algin. The methods may use multiple steps. In some instances, the step(s) includes applying a composition containing hydroxypropyl guar hydroxypropyltrimonium chloride, guar hydroxypropyltrimonium chloride, Argania spinosa kernel oil, and algin to the hair. The methods and compositions may repair split ends, such as 80% of split ends, such as equal to, more than, less than, a range of, or between 80, 81, 82, 83, 84, 85, 86, 87, 88, 89, 90, 91, 92, 93, 94, 95, 96, 97, or 98%, repair at least a portion of cracks in hair cuticles, protect against heat damage, reduce hair frizziness or hair fly-aways, increase the elongation at break of individual hair fibers, increase the tensile strength of the hair, reduce the loss of disulfide bonds in the hair at a temperature of up to 235° C., and/or forms at least two disulfide bonds from broken disulfide bonds of the hair with the bis-aminopropyl diglycol dimaleate and/or with the compound of Formula I.

These and other non-limiting aspects of the present invention are provided below.

### A. Active Agents

The compositions and methods using the compositions herein may contain one or more of the following active agents. The active agents in the composition is not limited to these active agents.

Hydroxypropyl guar hydroxypropyltrimonium chloride is a hydroxypropyl derivative of a quaternary ammonium derivative of guar gum that can be used on hair. Hydroxypropyl guar hydroxypropyltrimonium chloride is available from commercial sources.

Argania spinosa kernel oil is an oil from the argan kernel that can be used on hair. Argania spinosa kernel oil is available from commercial sources.

Guar hydroxypropyltrimonium chloride is a quaternary ammonium derivative of guar gum that can be used on hair. Guar hydroxypropyltrimonium chloride is available from commercial sources. In some instances, the guar hydroxypropyltrimonium chloride used or present in the composition(s) is from a commercial source such as PROVITAL™ under the tradename KERATRIX™ and/or SOLVAY™ under the tradename JAGUAR®C-17.

Algin is a colloidal substance typically derived from marine algae that can be used on hair. Algin is available from commercial sources.

Bis-aminopropyl diglycol dimaleate is a compound that can be used on hair and has been used on hair to restore and/or provide sulfur bridges of hair. In some instances, the compositions and/or methods herein replace the bis-aminopropyl diglycol dimaleate with, or use in addition to the bis-aminopropyl diglycol dimaleate, one or more of the compounds of Formula I that can restore and/or provide sulfur bridges of hair.

A fructooligosaccharide is a oligosaccharide fructan that can be used on hair. A fructooligosaccharide is available from commercial sources. In some instances, the fructooligosaccharide used or present in the composition(s) is from a commercial source such as CLR™ under the tradename MULTIMOIST™.

Beta vulgaris root extract is an extract from the roots of a beet plant that can be used on hair. Beta vulgaris root extract is available from commercial sources. In some instances, the Beta vulgaris root extract used or present in the composition(s) is from a commercial source such as CLR™ under the tradename MULTIMOIST™.

Arginine is an amino acid that can be used on hair. Arginine is available from commercial sources. In some instances, the arginine used or present in the composition(s) is from a commercial source such as AJINOMOTTO™ under the tradename PRODEW 500™.

Aspartic acid is an amino acid that can be used on hair. Aspartic acid is available from commercial sources. In some instances, the aspartic acid used or present in the composition(s) is from a commercial source such as AJINOMOTTO™ under the tradename PRODEW 500™.

Glycine is an amino acid that can be used on hair. Glycine is available from commercial sources. In some instances, the glycine used or present in the composition(s) is from a commercial source such as AJINOMOTTO™ under the tradename PRODEW 500™.

Alanine is an amino acid that can be used on hair. Alanine is available from commercial sources. In some instances, the alanine used or present in the composition(s) is from a commercial source such as AJINOMOTTO™ under the tradename PRODEW 500™.

Serine is an amino acid that can be used on hair. Serine is available from commercial sources. In some instances, the serine used or present in the composition(s) is from a commercial source such as AJINOMOTTO™ under the tradename PRODEW 500™.

Valine is an amino acid that can be used on hair. Valine is available from commercial sources. In some instances, the valine used or present in the composition(s) is from a commercial source such as AJINOMOTTO™ under the tradename PRODEW 500™.

Proline is an amino acid that can be used on hair. Proline is available from commercial sources. In some instances, the proline used or present in the composition(s) is from a commercial source such as AJINOMOTTO™ under the tradename PRODEW 500™.

Threonine is an amino acid that can be used on hair. Threonine is available from commercial sources. In some instances, the threonine used or present in the composition(s) is from a commercial source such as AJINOMOTTO™ under the tradename PRODEW 500™.

Isoleucine is an amino acid that can be used on hair. Isoleucine is available from commercial sources. In some instances, the isoleucine used or present in the composition(s) is from a commercial source such as AJINOMOTTO™ under the tradename PRODEW 500™.

Histidine is an amino acid that can be used on hair. Histidine is available from commercial sources. In some instances, the histidine used or present in the composition(s) is from a commercial source such as AJINOMOTTO™ under the tradename PRODEW 500™.

Phenylalanine is an amino acid that can be used on hair. Phenylalanine is available from commercial sources. In some instances, the phenylalanine used or present in the composition(s) is from a commercial source such as AJINOMOTTO™ under the tradename PRODEW 500™.

One or more additional active agents may be added to the haircare compositions. The combination of the active agents may have additive or synergistic effects.

### B. Additives

The hair compositions may include one or more of each of water, a sunscreen, a non-ionic, anionic, cationic, or zwitterionic surfactant, vitamins, pH adjusters, fragrance, solvents, chelating agents, preservatives, opacifying agents, volatiles, propellants, liquid vehicles, carriers, salts, buffers, emulsifying agents, softeners, fillers, moisturizers, hair fixatives, dyes, colorants, pigments, antifoaming agents, antioxidants, moisturizing agents, proteins, peptides, amino acids, extracts, resins, essential oils, thickening agents, viscosity modifiers, film formers, emollients, lubricants, butters, penetrants, anti-static agents, anti-frizz agents, anti-dandruff agents, absorbents, surfactants, detergents, pharmaceutically-active agents, vitamins, and other additives used by those skilled in the art.

### C. Surfactants

Surfactants may be amphoteric, anionic, or cationic. Exemplary surfactants include but are not limited to cocamidopropyl betaine, coco betaine, lauryl betaine, oleyl betaine, stearyl betaine, coco sultaine, lauryl sultaine, sodium cocoyl isethionate, sodium methyl 2-sulfolaurate, disodium 2-sulfolaurate, sodium lauryl sulfoacetate, ammonium lauryl sulfate, ammonium laureth sulfate, sodium lauryl sulfate, sodium laureth sulfate, sodium lauroyl sarcosinate, cocoyl sarcosine, sodium cocoyl lactylate, sodium cocoyl sarcosinate, 3-aminopropane sulfonic acid, almond amide, almond amidopropyl betaine, almond amidopropylamine oxide, aluminum hydrogenated tallow glutamate, aluminum lanolate, aminoethyl sulfate, aminopropyl lauryl glutamine, ammonium C12-15 alkyl sulfate, ammonium C12-15 pareth sulfate, ammonium C12-16 alkyl sulfate, ammonium C9-10 perfluoroalkylsulfonate, ammonium capryleth sulfate, ammonium capryleth-3 sulfate, ammonium monoglyceride sulfate, ammonium sulfate, ammonium isothionate, ammonium cocoyl sarcosinate, ammonium cumene sulfonate, ammonium dimethicone copolyol sulfate, ammonium dodecylbenzenesulfonate, ammonium isostearate, ammonium laureth-12 sulfate, ammonium laureth-5 sulfate, ammonium laureth-6 carboxylate, ammonium laureth-7 sulfate, ammonium laureth-8 carboxylate, ammonium laureth-9 sulfate, ammonium lauroyl sarcosinate, ammonium lauryl sulfosuccinate, ammonium myreth sulfate, ammonium myristyl sulfate, ammonium nonoxynol-30 sulfate, ammonium nonoxynol-4 sulfate, ammonium oleate, ammonium palm kernel sulfate, ammonium polyacrylate, ammonium stearate, ammonium tallate, ammonium xylene sulfonate, ammonium xylene sulfonate, amp-isostearoyl gelatin/keratin amino acids/lysine hydroxypropyltrimonium chloride, amp-isostearoyl hydrolyzed collagen, apricot kernel oil PEG-6 esters, apricot amide, apricot amidopropyl betaine, arachideth-20, avocadamide, avocadamidopropyl betaine, babassuamide, babassuamidopropyl betaine, babassuamidopropylamine oxide, behenalkonium chloride, behenamide, behenamide, behenamidopropyl betaine, behenamine oxide, a polyoxyether of lauryl alcohol or ceteareth-20, or combinations thereof.

Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer 401, stearoyl monoisopropanolamide, and polyoxyethylene hydrogenated tallow amide. Examples of amphoteric surfactants include sodium N-dodecyl-.beta.-alanine, sodium N-lauryl-β-iminodipropionate, myristoamphoacetate, lauryl betaine and lauryl sulfobetaine.

The surfactants are optionally included in an amount ranging from about 0.001% to about 20% by weight of the formulation, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, or 20 wt. %.

### D. Vitamins

Non-limiting examples of vitamins that may be added to the present compositions include niacinamide, sodium starch octenylsuccinate, calcium pantothenate, maltodextrin, sodium ascorbyl phosphate, pyridoxine HCl, silica, panthenol, phytantriol, calcium pantothenate, vitamin E, and vitamin E esters (e. g., tocopheryl acetate, tocopheryl nocotinate, tocopheryl palmitate, or tocopheryl retinoate).

### E. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

### F. pH Adjusters

Non-limiting examples of pH adjusters that may be used with the present compositions include common acids and bases, including but not limited to citric acid, acetic acid, benzoic acid, glycolic acid, lactic acid, malic acid, potassium hydroxide, potassium carbonate, sodium carbonate, sodium hydroxide, ethanolamine, and triethanolamine.

### G. Solvents

Non-limiting examples of solvents that may be included in the present ultraviolet light-absorbing compositions include but are not limited to water, propylene glycol, propane diol, glycerol, SD alcohol, mineral oil, butanediol, cyclomethicone, and ethoxyglycol.

### H. Preservatives

Non-limiting examples of preservatives include but are not limited to benzoic acid, benzoic acid salts, benzyl alcohol, butylparaben, propylparaben, methyparaben, isobutylparaben, methylisothiazolinone, methylchloroisothiazolinone, phenoxy-ethanol, quaterium-8, quaterium-14, quaterium-15, triclosan, zinc pyrithione, zinc salicylate, quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, glycerin containing compounds (e.g., glycerin or ethylhexylglycerin or phenoxyethanol), sodium benzoate, ethylenediamine-tetraacetic acid (EDTA), potassium sorbate, grapefruit seed extract, salicylic acid, DMDM hydantoin, formaldahyde, chlorphenism, triclosan, imidazolidinyl urea, diazolidinyl urea, sodium dehydroacetate, dehydroacetic acid, quaternium-15, stearalkonium chloride, sodium metabisulfite, 2-bromo-2-nitropropane, chlorhexidine digluconate, polyaminopropyl biguanide, benzalkonium chloride, sodium sulfite, sodium salicylate, citric acid, neem oil, essential oils (various), lactic acid, and vitamin E (tocopherol), or combinations thereof.

The preservatives are optionally included in an amount ranging from about 0.001% to about 7% by weight of the formulation, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, or 7 wt. %.

### I. Emulsifying Agents

Non-limiting examples of emulsifying agents that can be used with the present compositions include nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, copolymers of an unsaturated ester and styrene sulfonate monomer, ethoxylated ethers, ethoxylated alcohols, alkyl phosphates, polyoxyethylene fatty ether phosphates such as polyoxyethylene glycol ether of cetearyl alcohol, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, stearic acid, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, lecithin, glycol stearate, and mixtures thereof.

The emulsifying agents are optionally included in an amount ranging from about 0.001% to about 20% by weight of the formulation, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, or 20 wt. %.

### J. Antioxidants

Non-limiting examples of antioxidants that can be used with the compositions of the present invention include acetyl cysteine, ubiquinone, ascorbic acid polypeptide, ascorbyl dipalmitate, ascorbyl methylsilanol pectinate, ascorbyl palmitate, ascorbyl stearate, BHA, BHT, t-butyl hydroquinone, zinc, cysteine, cysteine HCl, diamylhydroquinone, di-t-butylhydroquinone, dicetyl thiodipropionate, dioleyl tocopheryl methylsilanol, lycopene, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, arbutin, nordihydroguaiaretic acid, octyl gallate, phenylthioglycolic acid, resveratrol, potassium ascorbyl tocopheryl phosphate, tetrahexydecyl ascorbate, potassium sulfite, propyl gallate, quinones, rosmarinic acid, sodium ascorbate, sodium bisulfite, sodium erythorbate, sodium metabisulfite, sodium sulfite, superoxide dismutase, carotenoids, sodium thioglycolate, sorbityl furfural, thiodiglycol, thiodiglycolamide, thiodiglycolic acid, thioglycolic acid, thiolactic acid, thiosalicylic acid, triethyl citrate, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

The antioxidant agents are optionally included in an amount ranging from about 0.001% to about 20% by weight of the formulation, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, or 20 wt. %.

### K. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

### L. Thickening Agents/Viscosity Modifying Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/vp copolymer, or a mixture of them.

Non-limiting examples of additional thickening agents that can be used in the present compositions include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium alginate, sodium carboxymethyl dextran, sodium carrageenan, tragacanth gum, xanthan gum, and mixtures thereof.

Viscosity modifying agents and/or additional thickening agents include, but are not limited to, viscous liquids, such as polyethylene glycol, semisynthetic polymers, such as semisynthetic cellulose derivatives, synthetic polymers, poloxamers, and polyethyleneimines (e.g., PEI-10), petroleum jelly, waxes, particulate associate colloids, such as bentonite and/or colloidal silicon dioxide, PPG-2 hydroxyethyl coco/isostearamide, disteareth-75 IPDI, and salts, such as sodium chloride, and combinations thereof.

### M. Emollients

Suitable emollients for use in the formulations include, but are not limited to a silicone compound (e.g., dimethicone, cyclomethicone, dimethicone copolyol or a mixture of cyclopentasiloxane and dimethicone/vinyldimethicone cross polymer, cyclopentasiloxane polysilicone), polyols such as sorbitol, glycerin, propylene glycol, ethylene glycol, polyethylene glycol, caprylyl glycol, polypropylene glycol, 1,3-butane diol, hexylene glycol, isoprene glycol, xylitol; ethylhexyl palmitate; a triglyceride such as caprylic/capric triglyceride and fatty acid ester such as cetearyl isononanoate or cetyl palmitate. In a specific embodiment, the emollient is dimethicone, amidodimethicone, dimethiconol, cyclopentasiloxane, potassium dimethicone PEG-7 panthenyl phosphate, or a combination thereof. In some instances, the composition(s) do not contain a silicone compound.

The emollients are optionally included in an amount ranging from about 0.001% to about 20% by weight of the formulation, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, or 20 wt. %.

### N. Conditioning Agents

Conditioning agents include, but are not limited to, silicone-based agents (e.g., silicone quaternium-8), panthenol, hydrolyzed wheat and/or soy protein, amino acids (e.g. wheat amino acids), rice bran wax, meadowfoam seed oil, mango seed oil, grape seed oil, jojoba seed oil, sweet almond oil, hydroxyethyl behenamidopropyl dimonium chloride, aloe leaf extract, aloe barbadensis leaf juice, phytantriol, panthenol, retinyl palmitate, behentrimonium methosulfate, cyclopentasiloxane, quaternium-91, stearamidopropyl dimethylamine, cationic polymers derived from polysaccharides, for example cationic cellulose derivatives, cationic starch derivatives, cationic guar derivatives and cationic locust bean gum derivatives, synthetic cationic polymers, mixtures or combinations of these agents. The formulation may comprise other synthetic or natural polymers or polymers derived from biological preparation processes, which are functionalized, where appropriate, for example with cationic or neutral groups. These polymers may have a stabilizing or strengthening action on the compositions, and/or a conditioning action (deposition on the surface of the skin or the hair), and combinations thereof. In some instances, the composition(s) do not contain a silicone compound.

The conditioning agents are optionally included in an amount ranging from about 0.001% to about 60% by weight of the formulation, such as equal to, more than, less than, a range of, or between 0.001, 0.005, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, or 60 wt. %.

### O. Opacifying Agents

Opacifying agents include, but are not limited to, glycol distearate and ethoxylated fatty alcohols.

### P. Propellant

Propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed.

The amount of propellant may range from about 10% to about 60% by weight of the formulation such as equal to, more than, less than, a range of, or between 10, 11, 12, 13, 14, 15, 16, 17, 18, 19, 20, 21, 22, 23, 24, 25, 26, 27, 28, 29, 30, 35, 40, 45, 50, 55, or 60 wt. %.

The propellant may be separated from the hair formulation as in a two compartment container. Other suitable aerosol dispensers are those characterized by the propellant being compressed air, which can be filled into the dispenser using a pump or equivalent device prior to use. Conventional non-aerosol pump spray dispensers, i.e., atomizers, may also be used to apply the hair strengthening formulation to the hair.

### Q. Pharmaceutically-Active Agents

Pharmaceutically-active agents are also contemplated as being useful with the present compositions. Non-limiting examples of pharmaceutical active agents include antibiotics, antifungals, antivirals, antimicrobials, biologically active proteins and peptides, depigmenting agents, hair growth stimulants, hair growth retardants including DFMO and its salts and analogs, steroids including hormones and corticosteroids, etc.

### R. Amounts of Ingredients

It is contemplated that the compositions of the present invention can include any amount of the ingredients discussed in this specification. The compositions can also include any number of combinations of additional ingredients described throughout this specification (e.g., pigments, or conditioning agents or pharmaceutical ingredients). The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### S. Carriers

The compositions of the present invention can include or be incorporated into all types of carriers and vehicles. The carrier or vehicle can be a pharmaceutically or dermatologically acceptable carrier or vehicle. Non-limiting examples of carriers or vehicles include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

### T. Formulations

The compositions of the present invention can be structured or formulated into a variety of different forms. Non-limiting examples include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as powders), gels, masks, and ointments. Variations and other structures will be apparent to the skilled artisan and are appropriate for use in the present invention.

### U. Method of Use

In one embodiment, prior to treatment with the methods and compositions described herein, the hair has been damaged, such as hair that at least some of the thiol groups in the hair are free thiols. The methods and compositions can be applied to the hair to bind the free thiol groups, to treat damage to hair, and/or prevent damage to the hair. In some instances, the methods and/or compositions are applied at least within one week of the hair being damaged, preferably within three days, more preferably within two days, most preferably, the same day. In some instances, the methods and/or compositions are applied to protect the hair from damage within one week of the hair being damaged, preferably within three days, more preferably within two days, most preferably, the same day.

### 1. Rinse or Wash the Hair

Optionally, the hair may be shampooed and/or conditioned prior to applying the methods and/or compositions. Alternately, the hair may be dry or may only be rinsed with water prior to application of the methods and/or compositions.

2. Apply the Methods and/or Compositions to the Hair

In some instances, subsequent to shampooing, conditioning, and/or rinsing the hair, the method and/or composition is applied to the hair. Alternately, the hair does not have to be washed or rinsed prior to application of the method and/or composition. In some embodiments, the method and/or composition is applied to dry hair.

The compositions may be used on hair daily, weekly, monthly, yearly or any amount of time therein.

Typically, the amount of compositions applied is sufficient to lightly coat the hair.

The methods and/or compositions may be applied to the hair as a single application, or application may be repeated one or more times. Typically, the amount of composition applied in each application is sufficient to lightly coat the hair. The volume of the composition(s) applied to the hair in each application may be about 1 to about 100 mL per person depending on their length and volume of hair, such as equal to, more than, less than, a range of, or between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ml. In some embodiments, application of the composition could be repeated immediately (e.g. within about 10 to 15 seconds) or between about one and five minutes, greater than five minutes, between about five and ten minutes, greater than ten minutes, between about ten and twenty (20) minutes after the first application, such as equal to, more than, less than, a range of, or between 1, 2, 3, 4, 5, 6, 7, 8, 9, 10, 11, 12, 13, 14, 15, 16, 17, 18, 19, or 20 minutes.

3. Remove the Composition(s) from the Hair

In some instances, the hair does not have to be washed or rinsed subsequent to application of the compositions and/or methods. In some instances, the composition is not completely and/or partially rinsed and/or washed from the hair before styling the hair. In some aspects, the styling includes heating the hair.

Alternatively, the hair is washed or rinsed subsequent to the application of the methods and/or compositions after the treatment period is over if a treatment period is used. The hair may be rinsed and subsequently washed immediately (e.g. within 10, 15, 25, 30, 45, 60 seconds (one minute), two minutes, three minutes, four, or five minutes following application) after final application of the composition(s). Alternatively the hair may be rinsed and/or washed within about 3 hours following application, such as 5 minutes, 10 minutes, 15 minutes, 20 minutes, 25 minutes, 30 minutes, 35 minutes, 40 minutes, 45 minutes, 50 minutes, 55 minutes, 1 hour, 1 hour and 5 minutes, 1 hour and 10 minutes, 1 hour and 15 minutes, 1 hour and 20 minutes, 1 hour and 25 minutes, 1 hour and 30 minutes, 1 hour and 35 minutes, 1 hour and 40 minutes, 1 hour and 45 minutes, 1 hour and 50 minutes, or 1 hour and 55 minutes, 2 hours, 2 hours and 5 minutes, 2 hours and 10 minutes, 2 hours and 15 minutes, 2 hours and 20 minutes, 2 hours and 25 minutes, 2 hours and 30 minutes, 2 hours and 35 minutes, 2 hours and 40 minutes, 2 hours and 45 minutes, 2 hours and 50 minutes, 2 hours and 55 minutes, or 3 hours.

In some instances, the composition covalently binds latent free thiols in the hair. The thiols remain bound for at least 1, 2, 3, 4, 5, or 6 days, or 1, 2, 3, 4, 5, 6, 7, 8, 8, 10, 11, or 12 weeks. The binding reaction is a stable reaction, such that the thiols may remain bound even if subjected to a hair coloring treatment (simultaneous or subsequent to the binding reaction).

4. Chemical Treatment of Hair with a Reducing Agent

In one embodiment, prior to treatment with the method and/or composition, the hair has been subjected to a reducing agent used for waving (also referred to herein as hair perming or permanent waves), curling, and/or straightening of the hair.

a. Apply a Reducing Agent to the Hair

The first step in waving, curling, or straightening hair may be breaking the cysteine disulfide bonds to form free thiol moieties. The process for breaking the cysteine disulfide bonds is via application of a reducing agent. The process for applying the reducing agent involves following normal perming or hair straightening procedures, that are known to those skilled in the art. For example, to perm a hair, the hair is first washed and set on perm rods of various sizes. Second, a reducing agent, such as thioglycolate reducing solution or lotion, is applied to the hair. The hair is allowed to set for a specified period of time, and then the thioglycolate solution is rinsed from the hair.

The application of hydrogen peroxide in the process of waving, curling, or straightening is optional. In some processes, such as when treating previously chemically treated hair, hydrogen peroxide is generally not used. In other processes, such as when perming virgin hair, hydrogen peroxide may be added. In these embodiments, hydrogen peroxide is typically added after the reducing agent is rinsed out. Then the hydrogen peroxide typically rinsed from the hair prior to the method and/or composition.

b. Apply the Method(s) and/or Composition(s)

Subsequent to the reducing treatment, the composition is applied to the hair. Although the methods and/or compositions are typically applied on the same day as treatment with the reducing agent, they may be applied later, such as within 1 to 2 weeks following treatment with the reducing agent.

In some instances, the hair does not have to be washed or rinsed subsequent to application of the compositions and/or methods. In some instances, the composition is not completely rinsed and/or washed from the hair before styling the hair. In some instances, styling the hair includes heating the hair.

Alternately, the hair is washed or rinsed subsequent to the application of the methods and/or compositions after the treatment period is over if a treatment period is used. The hair may be rinsed and subsequently washed immediately (e.g. within 10, 15, 25, 30, 45, 60 seconds (one minute), two minutes, three minutes, four, or five minutes following application) after final application of the composition(s). Alternatively the hair may be rinsed and/or washed within about 3 hours following application, such as 5 minutes, 10 minutes, 15 minutes, 20 minutes, 25 minutes, 30 minutes, 35 minutes, 40 minutes, 45 minutes, 50 minutes, 55 minutes, 1 hour, 1 hour and 5 minutes, 1 hour and 10 minutes, 1 hour and 15 minutes, 1 hour and 20 minutes, 1 hour and 25 minutes, 1 hour and 30 minutes, 1 hour and 35 minutes, 1 hour and 40 minutes, 1 hour and 45 minutes, 1 hour and 50 minutes, or 1 hour and 55 minutes, 2 hours, 2 hours and 5 minutes, 2 hours and 10 minutes, 2 hours and 15 minutes, 2 hours and 20 minutes, 2 hours and 25 minutes, 2 hours and 30 minutes, 2 hours and 35 minutes, 2 hours and 40 minutes, 2 hours and 45 minutes, 2 hours and 50 minutes, 2 hours and 55 minutes, or 3 hours.

The compositions described herein improve hair quality, such as appearance (e.g., sheen) and feel, and decrease hair breakage without decreasing strength (e.g., tensile strength) when the hair is subjected to subsequent treatments and/or styling, such as coloring and/or heat treatments.

In some embodiments, hair breakage decreases by 5, 10, 15, 20, 25, 30, 35, 40, 45, 50, 55, 60, 65, 70, or 75% or higher after treatment with the method(s) and/or composition(s) compared to untreated hair from the same individual. Hair breakage is a significant problem encountered during coloring, heating, and other treatments.

### 5. Apply Coloring Formulation to the Hair

In some instances, the methods and/or compositions are applied to color treated and/or colored hair, such as dyed hair. In some instances, the hair is color treated and/or colored subsequent to the application of the method(s) and/or composition(s).

Coloring formulation are generally applied to an individual's hair following normal hair coloring procedures that are known to those skilled in the art. Typically, hair color treatments include two complementary processes: bleaching the hair's natural pigment and/or other artificial pigments present in the hair, and diffusion of dye precursors into the hair, followed by coupling reactions that result in the formation of chromophores within the hair shaft, which are too large to diffuse out of the hair. The hair coloring formulation may be a highlighting formulation, such as formed by mixing bleach powder and developer. More complex colors may contain several precursors and many couplers, and may involve multiple reactions.

The dye precursors may contain several ingredients, each with different functions. The first ingredient is usually an alkalizing agent (usually ammonia and/or an ammonia substitute, such as monoethanolamine [MEA]). The alkalizing agent serves a number of roles in the hair colorant process including swelling the hair fiber to aid in diffusion of the dye precursors. The dye precursors generally include p-diamines and p-aminophenols. Precursors are oxidized to active intermediates once they have penetrated the hair shaft. Intermediates then react with color couplers to create wash resistant dyes. More specifically, the intermediates, in the presence of an oxidant, couple with another oxidation dye intermediate molecule to form a large fused ring color compound within the hair shaft. The precursor intermediate should penetrate the hair shaft prior to the coupling reaction since the fused ring product is too large to penetrate the hair shaft. Couplers modify the color produced by the oxidation of precursor compounds. The primary difference between demi-permanent and permanent products is the alkalizing agent and the concentration of peroxide. The cuticle does not swell as greatly with demi-permanent dyes, making dye penetration less efficient compared to permanent coloring products.

Several coloring formulations use a reducing agent, such as sodium bisulfate, to break disulfide bonds in the hair, allowing deeper penetration of the hair coloring dyes into the hair. Specifically, the method includes reducing some of the disulfide linkages of the cysteine in the hair shafts to thiol groups while breaking hydrogen bonds. The reducing process changes the chemical and cosmetic characteristics of the hair, which are undesirable.

The hair dyeing process may be followed by a shampoo and conditioning treatment, a neutralizing rinse or an acid balanced shampoo containing in addition to cationic or amphoteric surfactants, cation-active emollients and quaternary polymers. Alternately, the hair dyeing process may be followed by application of the method(s) and/or composition(s) described herein, before and/or after a shampoo and/or conditioning treatment.

a. Applying Binding Formulation

The method(s) and/or composition(s) may be applied simultaneously with hair coloring formulation or subsequent to the application of the hair coloring formulation. For example, the composition may be mixed with the hair coloring treatment and the mixture, containing both the composition and the hair coloring treatment, may be applied to the hair.

Alternatively, subsequent to coloring the hair, the method(s) and/or composition(s) is/are applied to the hair. Although the methods and/or compositions are typically applied on the same day as coloring the hair, they may be applied later, such as within 1 to 2 weeks following coloring the hair.

In some instances, the hair is washed or rinsed subsequent to the coloring of the hair and before application of the methods and/or compositions. The hair may be rinsed and subsequently washed immediately (e.g., within 10, 15, 25, 30, 45, 60 seconds (one minute), two minutes, three minutes, four, or five minutes following application) after final application of the composition(s). Alternatively the hair may be rinsed and/or washed within about 3 hours following application, such as 5 minutes, 10 minutes, 15 minutes, 20 minutes, 25 minutes, 30 minutes, 35 minutes, 40 minutes, 45 minutes, 50 minutes, 55 minutes, 1 hour, 1 hour and 5 minutes, 1 hour and 10 minutes, 1 hour and 15 minutes, 1 hour and 20 minutes, 1 hour and 25 minutes, 1 hour and 30 minutes, 1 hour and 35 minutes, 1 hour and 40 minutes, 1 hour and 45 minutes, 1 hour and 50 minutes, or 1 hour and 55 minutes, 2 hours, 2 hours and 5 minutes, 2 hours and 10 minutes, 2 hours and 15 minutes, 2 hours and 20 minutes, 2 hours and 25 minutes, 2 hours and 30 minutes, 2 hours and 35 minutes, 2 hours and 40 minutes, 2 hours and 45 minutes, 2 hours and 50 minutes, 2 hours and 55 minutes, or 3 hours.

Alternately, the hair does not have to be washed or rinsed subsequent to coloring the hair.

The compositions described herein improve hair quality, such as appearance (e.g., sheen) and feel and decrease hair breakage without decreasing dry strength (e.g., tensile strength) when the hair is subjected to subsequent treatments, such as coloring and/or heating.

In some embodiments, hair breakage decreases by 5, 10, 15, 20, 25, 30, 35, 40, 45, 50, 55, 60, 65, 70, or 75% or higher after treatment with the method(s) and/or composition(s) compared to untreated hair from the same individual. Hair breakage is a significant problem encountered during coloring, heating, and other treatments.

If the composition is combined with the hair coloring treatment and applied as a mixture to the hair, then the mixture remains on the hair as long as needed for the hair coloring treatment. Typically, the mixture is applied for approximately 10 minutes. The mixture is removed from the hair in accordance with standard methods for hair coloring treatments, e.g., rinse and shampoo, approximately 10 minutes after applying the mixture.

The composition(s) may be rinsed from the hair after application. The hair may be rinsed and subsequently washed immediately (e.g., within 10 to 15 seconds following application) after final application of the composition(s). In some instances, the hair is rinsed and/or washed about 10 minutes or later after the final application of the composition(s), such as about 15 minutes to about 30 minutes.

### V. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, or other types of containers such as injection or blow-molded plastic containers. The kit and/or container can include indicia on its surface. The indicia, for example, can be a word, a phrase, an abbreviation, a picture, or a symbol. In some instances, the kit can include two ore more containers with different compositions in each container to implement a multi-step processes of the present invention.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### A. Example 1 (Exemplary Formulations)

Formulations were prepared as hair compositions. The formulation in Table 1 is an example of hair care product. The formulation in Table 2 is an example of a second hair care product. The formulation in Table 3 is an example of a third hair care product. The formulation in Table 4 is an example of a fourth hair care product. The formulation in Table 5 is an example of a leave in conditioner. The formulation in Table 6 is an example of a second leave in conditioner.

TABLE 1

| | % Concentration |
|---|---|
| Ingredient | (by weight) |
| Hydroxypropyl Guar Hydroxypropyltrimonium | 0.25 to 0.75 |
| Chloride | |
| Argania spinosa Kernel Oil | 0.5 to 0.5 |
| Guar Hydroxypropyltrimonium Chloride | 0.1 to 0.5 |
| Algin | 0.001 to 0.2 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 70% w/w, and preferably between 70 to 99% w/w.

TABLE 2

| | % Concentration |
|---|---|
| Ingredient | (by weight) |
| Hydroxypropyl Guar Hydroxypropyltrimonium | 0.25 to 0.75 |
| Chloride | |
| Argania spinosa Kernel Oil | 0.5 to 0.5 |
| Guar Hydroxypropyltrimonium Chloride | 0.1 to 0.5 |
| Algin | 0.001 to 0.2 |
| Bis-Aminopropyl Diglycol Dimaleate | 0.001 to 0.2 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 70% w/w, and preferably between 70 to 99% w/w.

TABLE 3

| | % Concentration |
|---|---|
| Ingredient | (by weight) |
| A Fructooligosaccharide | 0.1 to 2 |
| Beta vulgaris Root Extract | 0.1 to 1.5 |
| Hydroxypropyl Guar Hydroxypropyltrimonium | 0.25 to 0.75 |
| Chloride | |
| Argania spinosa Kernel Oil | 0.5 to 0.5 |
| Guar Hydroxypropyltrimonium Chloride | 0.1 to 0.5 |
| Algin | 0.001 to 0.2 |
| Bis-Aminopropyl Diglycol Dimaleate | 0.001 to 0.2 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 70% w/w, and preferably between 70 to 98% w/w.

TABLE 4

| | % Concentration |
|---|---|
| Ingredient | (by weight) |
| A Fructooligosaccharide | 0.1 to 2 |
| Beta vulgaris Root Extract | 0.1 to 1.5 |
| Arginine | 0.01 to 1 |
| Hydroxypropyl Guar Hydroxypropyltrimonium | 0.25 to 0.75 |
| Chloride | |
| Argania spinosa Kernel Oil | 0.5 to 0.5 |
| Guar Hydroxypropyltrimonium Chloride | 0.1 to 0.5 |
| Aspartic Acid | 0.01 to 0.5 |
| Algin | 0.001 to 0.2 |
| Bis-Aminopropyl Diglycol Dimaleate | 0.001 to 0.2 |
| Glycine | 0.001 to 0.1 |
| Alanine | 0.001 to 0.1 |
| Serine | 0.001 to 0.1 |
| Valine | 0.001 to 0.1 |
| Proline | 0.0001 to 0.1 |
| Threonine | 0.0001 to 0.1 |
| Isoleucine | 0.0001 to 0.1 |
| Histidine | 0.0001 to 0.1 |
| Phenylalanine | 0.0001 to 0.1 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 70% w/w, and preferably between 70 to 98% w/w.

TABLE 5

| | % Concentration |
|---|---|
| Ingredient | (by weight) |
| A Fructooligosaccharide | 0.1 to 2 |
| Phenoxyethanol | 0.1 to 2 |
| Beta vulgaris Root Extract | 0.1 to 1.5 |
| Sodium Lactate | 0.1 to 1 |
| Caprylic/Capric Triglyceride | 0.1 to 1 |
| Potassium Lactate | 0.01 to 1 |
| Sodium Pyrrolidone Carboxylic Acid | 0.01 to 1 |
| Arginine | 0.01 to 1 |
| Hydroxypropyl Guar Hydroxypropyltrimonium | 0.25 to 0.75 |
| Chloride | |
| Argania spinosa Kernel Oil | 0.5 to 0.5 |
| Guar Hydroxypropyltrimonium Chloride | 0.1 to 0.5 |
| Aspartic Acid | 0.01 to 0.5 |
| Pyrrolidone Carboxylic Acid | 0.01 to 0.5 |
| Lactic Acid | 0.001 to 0.5 |
| Algin | 0.001 to 0.2 |
| Bis-Aminopropyl Diglycol Dimaleate | 0.001 to 0.2 |
| Sorbitan Oleate | 0.001 to 0.2 |
| Coco-Glucoside | 0.001 to 0.2 |
| Carrageenan | 0.001 to 0.1 |
| Xanthan gum | 0.001 to 0.1 |
| Chitosan | 0.001 to 0.1 |
| Glycine | 0.001 to 0.1 |
| Alanine | 0.001 to 0.1 |
| Serine | 0.001 to 0.1 |
| Valine | 0.001 to 0.1 |
| Proline | 0.0001 to 0.1 |
| Threonine | 0.0001 to 0.1 |
| Isoleucine | 0.0001 to 0.1 |
| Histidine | 0.0001 to 0.1 |
| Phenylalanine | 0.0001 to 0.1 |
| Sodium Benzoate | 0.00001 to 0.01 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 70% w/w, and preferably between 70 to 98% w/w.

TABLE 6

| | % Concentration |
|---|---|
| Ingredient | (by weight) |
| A Fructooligosaccharide | 0.1 to 2 |
| Phenoxyethanol | 0.1 to 2 |
| Beta vulgaris Root Extract | 0.1 to 1.5 |
| Sodium Lactate | 0.1 to 1 |
| Caprylic/Capric Triglyceride | 0.1 to 1 |
| Potassium Lactate | 0.01 to 1 |
| Sodium Pyrrolidone Carboxylic Acid | 0.01 to 1 |
| Arginine | 0.01 to 1 |
| Hydroxypropyl Guar Hydroxypropyltrimonium | 0.25 to 0.75 |
| Chloride | |
| Argania spinosa Kernel Oil | 0.5 to 0.5 |
| Guar Hydroxypropyltrimonium Chloride | 0.1 to 0.5 |
| Aspartic Acid | 0.01 to 0.5 |
| Pyrrolidone Carboxylic Acid | 0.01 to 0.5 |
| Lactic Acid | 0.001 to 0.5 |
| Algin | 0.001 to 0.2 |
| Bis-Aminopropyl Diglycol Dimaleate | 0.001 to 0.2 |
| Sorbitan Oleate | 0.001 to 0.2 |
| Coco-Glucoside | 0.001 to 0.2 |
| Carrageenan | 0.001 to 0.1 |
| Xanthan gum | 0.001 to 0.1 |
| Chitosan | 0.001 to 0.1 |
| Glycine | 0.001 to 0.1 |
| Alanine | 0.001 to 0.1 |
| Serine | 0.001 to 0.1 |
| Valine | 0.001 to 0.1 |
| Proline | 0.0001 to 0.1 |
| Threonine | 0.0001 to 0.1 |
| Isoleucine | 0.0001 to 0.1 |
| Histidine | 0.0001 to 0.1 |
| Phenylalanine | 0.0001 to 0.1 |
| Sodium Benzoate | 0.00001 to 0.01 |
| Water | 70 to 98 |
| Cetearyl Alcohol | 1 to 5 |
| Astrocaryum murumuru Seed Butter | 1 to 5 |
| Propanediol | 0.5 to 3 |
| Behentrimonium Chloride | 0.5 to 3 |
| Glycerin | 0.1 to 3 |
| Cetyl Alcohol | 0.1 to 3 |
| Isoamyl Laurate | 0.1 to 3 |
| Squalane | 0.1 to 3 |
| Brassicyl Valinate Esylate | 0.1 to 2 |
| Cetyl Esters | 0.1 to 2 |
| Pentylene Glycol | 0.1 to 2 |
| Phenethyl Benzoate | 0.1 to 2 |
| Brassica campestris/Aleurites fordi Oil Copolymer | 0.1 to 1.5 |
| Cetrimonium Chloride | 0.1 to 1.5 |
| Caesalpinia spinosa Gum | 0.1 to 1.5 |
| Hydroxypropyl Starch Phosphate | 0.01 to 1 |
| Hydrogenated Ethylhexyl Olivate | 0.01 to 1 |
| Calcium Gluconate | 0.01 to 1 |
| Caprylyl Glycol | 0.01 to 1 |
| Cocos nucifera (Coconut) Oil | 0.01 to 1 |
| Panthenol | 0.01 to 1 |
| Ethylhexylglycerin | 0.01 to 1 |
| Oryza sativa (Rice) Bran Extract | 0.01 to 1 |
| Sodium Phytate | 0.01 to 1 |
| Ximenia americana Seed Oil | 0.01 to 1 |
| Excipients** | q.s. |

*Formulation can be prepared by mixing the ingredients in a beaker under heat 70-75° C. until homogenous. Subsequently, the formulation can be cooled to standing room temperature (20-25° C.). Further, and if desired, additional ingredients can be added, for example, to modify the rheological properties of the composition.
**Excipients can be added, for example, to modify the rheological properties of the composition. Alternatively, the amount of water can be varied so long as the amount of water in the composition is at least 70% w/w, and preferably between 70 to 98% w/w.

### B. Example 2 (Testing on Hair)

A non-limiting example of a method disclosed herein using non-limiting examples of a composition disclosed herein. It was determined that the method does not change the dry color of dyed hair, seals split ends, seals cuticle cracks, smooths the hair, decreases the amount of hair breakage, and protects the hair from heat damage, decreases the frizzing of hair, and decreases the amount of knots formed in hair/the friction when combing wet hair.

Color Retention: Hair was tested to demonstrate the manner by which the treatment method using the composition changes the color of hair with repeated washing via color testing using a Hunterlab Colorimeter.

3 g, 8″ long, 1″ wide Curly multi-ethnic brown tresses (Walker type 3A), 6% bleached and colored with commercially available auburn dye and 3 g, 8″ long, 1″ wide European medium brown tresses, 6% bleached and colored with commercially available auburn dye were tested, ten tresses per treatment group. All of the hair was first bleached by contacting the hair with a skeletal 6% hydrogen peroxide solution that has a pH of 10.2 and a temperature of 40° C. at 500 ml per 40 tresses for 40 minutes at 40° C. with regular agitation and afterwards rinsed twice with water and then placed under running water. All of the tresses were dyed with L'Oréal Preference shade 4R Dark Auburn dye. The color components were combined using the manufacturer's instructions and mixed. Then 10 g of the mixture was applied to each tress using a brush (5 g on each side of the tress) and massaged thoroughly into the tress. The hair was maintained at 27° C. for 20 minutes and massaged at 10 minutes into the 20 minute treatment. The color treated tresses were rinsed out under running water at a temperature of 40° C. for 2 minutes or until the water ran clear. The rinsed hair was allowed to rest for at least 12 hours prior to taking post color treatment readings.

The tresses were divided into two treatment groups, a Method Treated group and a Control group. The Method Treated group tresses were 1) washed with a sodium laureth sulfate (SLES) solution containing 15% SLES, the solution being used at 10% by weight of the tresses for the shampoo (0.1 ml/g of hair), massaged for 30 seconds, and then rinsed for 30 seconds, 2) the composition of Table 6 was applied to the damp hair at 10% by weight of the tresses (0.1 ml/g of hair), massaged for 30 seconds, and blow dried on the “no heat” setting. If a further wash and treatment cycle was performed, steps 1) and 2) were repeated. A Control group of tresses were washed and treated as described for the Method Treated procedure but used water instead of the composition of Table 6.

Color was measured after drying and equilibrating over-night at 60% relative humidity (RH) & 22° C. after each of the initial dying, after a first wash and treatment after dying, after five washes and treatments after dying, and after ten washes and treatments after dying. For the color measurements, CIELAB L, a, and b measurements were taken using a Hunter Lab UltraScan VIS colorimeter. “L” refers to the lightness on a scale of 0 to 100, “a” denotes the red-green color range (positive value denotes higher red), and “b” represents the yellow-blue color range (positive value denotes higher yellow). Changes in the color of the tress were calculated using the difference in these parameters relative to the baseline measurement after treatment (e.g., ΔL*, Δa*, and Δb*). Overall color change, ΔE was calculated using the following Equation 1.

$$Δ E = √ [ Δ {L}^{* 2} + Δ {a}^{* 2} + Δ {b}^{* 2} ] \qquad Equation 1$$

Ten color measurements were performed on each tress (with ten tresses per treatment). JMP™ analytical software was used to calculate the statistics (student's t-test at 95% confidence level).

For the European medium brown tresses, after the first wash and treatment after dying, the Method Treated hair showed a statistically higher change in color when compared to Control, but the difference in mean measurements was less than 2, which is not visible to the human eye. After the fifth wash and treatment after dying, the Control showed a statistically higher change in color when compared to Method Treated hair, but the difference in mean measurements was less than 2, which is not visible to the human eye. At ten washes, there was no statistical difference between Control and the Method Treated hair. This indicates that the method treatment is safe for color-treated hair.

For the Curly multi-ethnic brown tresses, after the first wash and treatment after dying, there was no statistical difference between Control and the Method Treated hair. After the fifth wash and treatment after dying, the Control showed a statistically higher change in color when compared to Method Treated hair, but the difference in mean measurements was less than 2, which is not visible to the human eye. At ten washes, the Method Treated hair showed a statistically higher change in color when compared to Control, but the difference in mean measurements was less than 2, which is not visible to the human eye.

Together, these indicate that the Method Treatment and leave-in conditioner composition is safe for color-treated hair.

Hair Breakage: Hair was tested to demonstrate the ability of the treatment method using the composition to protect hair against breakage via repeated grooming.

3 g, 8″ long, 1″ wide Curly multi-ethnic brown tresses (Walker type 3A), 9% bleached (Curly) and 3 g, 8″ long, 1″ wide European medium brown tresses, 9% bleached (European) were tested, ten tresses per treatment group. All of the hair was first bleached three times by contacting the hair with a 9% hydrogen peroxide solution that has a pH of 10.2 and a temperature of 40° C. for 20 minutes at 40° C. with regular agitation and rinsed under running water between repeated bleaching and after the third bleaching.

The tresses were divided into three treatment groups, a 1× Method Treated group, a 3× Method Treated group, and a Control group. The 1× Method Treated group tresses were 1) washed with a sodium laureth sulfate (SLES) solution containing 15% SLES, the solution being used at 10% by weight of the tresses for the shampoo, by applying the shampoo at 10% by weight of the tresses (0.1 ml/g of hair), massaged for 30 seconds, and then rinsed for 30 seconds, 2) the composition of Table 6 was applied to the damp hair at 10% by weight of the tresses (0.1 ml/g of hair), massaged for 30 seconds, and allowed to air dry for 48 hours at 60% relative humidity. The 3× Method Treated group followed the method for the 1× Method Treated group but before drying repeated twice steps 1) and 2) for a total of three composition application cycles before drying. A Control group of tresses were washed as described for the 1× Method Treated procedure but not further treated before drying as described for the 1× Method Treated group.

Determining whether the treatment will protect hair against breakage was tested by repeated grooming experiments. The number of broken fibers was recorded as a function of repeated combing/brushing strokes. A treatment that reduces snags, entanglements, and abrasion can lower the number of broken fibers. Hair breakage by dry grooming was measured by using an automatic groomer for a total of 2,000 cycles. Fibers were collected and counted after every 200 cycles. Eight replicates for each sample were performed. The methodology used has been documented in the scientific literature (Evans & Park, A Statistical Analysis of Hair Breakage. II. Repeated Grooming Experiments, J. Cosmet. Sci., 61, 439-455, 2010). JMP™ analytical software was used to calculate the statistics (student's t-test at 95% confidence level). The percentage reduction in breakage was calculated using the following Equation 2.

$$\% Reduction in breakage = ( 1 - ( Breakage of Method Treated / Breakage of Control ) ) \times 100 \qquad Equation 2$$

A statistical reduction in breakage was seen for the 1× Method Treated group and 3× Method Treated group compared to the Control group, with the 1× Method Treated showing a 73% reduction in breakage of the European hair and a 81% reduction in breakage of the Curly Multi-ethnic hair, and with the 3× Method Treated showing a 77% reduction in breakage of the European hair and a 84% reduction in breakage of the Curly Multi-ethnic hair. See Table 7.

TABLE 7

| Group | N | Mean | Std. Dev. | Std. Err. Mean |
|---|---|---|---|---|
| Control - | 10 | 60 | 21 | 7 |
| European | | | | |
| 1 x Method | 10 | 16* | 6 | 2 |
| Treated - | | | | |
| European | | | | |
| 3x Method | 10 | 14* | 7 | 2 |
| Treated - | | | | |
| European | | | | |
| Control - Curly | 10 | 85 | 23 | 7 |
| 1x Method | 9 | 16* | 6 | 2 |
| Treated - Curly | | | | |
| 3x Method | 10 | 14* | 7 | 2 |
| Treated - Curly | | | | |

*statistically different from the respective Control

Wet Combing: Hair was tested to demonstrate the ability of the treatment method using the composition to smooth damaged hair via wet combing by the Garcia Method.

3 g, 8″ long, 1″ wide Curly multi-ethnic brown tresses (Walker type 3A), 6% bleached (Curly) and 3 g, 8″ long, 1″ wide European Medium Brown tresses, 6% bleached were tested, eight tresses per treatment group were tested. All of the hair was first bleached by contacting the hair with a skeletal 6% hydrogen peroxide solution that has a pH of 10.2 and a temperature of 40° C. at 500 ml per 40 tresses for 40 minutes at 40° C. with regular agitation and afterwards rinsed twice with water and then placed under running water.

The tresses were divided into three treatment groups, a 1× Method Treated group, a 3× Method Treated group, and a Control group. The 1× Method Treated group tresses were 1) washed with a sodium laureth sulfate (SLES) solution containing 15% SLES, the solution being used at 10% by weight of the tresses for the shampoo, by applying the shampoo at 10% by weight of the tresses (0.1 ml/g of hair), massaged for 30 seconds, and then rinsed for 30 seconds, 2) the composition of Table 6 was applied to the damp hair at 10% by weight of the tresses (0.1 ml/g of hair), massaged for 30 seconds, and allowed to air dry for 48 hours at 60% relative humidity. The 3× Method Treated group followed the method for the 1× Method Treated group but before drying repeated twice steps 1) and 2) for a total of three composition application cycles before drying. A Control group of tresses were washed as described for the 1× Method Treated procedure but not further treated before drying as described for the 1× Method Treated group.

Determining whether the treatment will smooth the hair was tested by an Instron tensile tester to measure frictional forces while a wet hair tress is pulled through a comb. A treatment that smooths hair can lower the frictional forces. Six replicates for each sample were performed. The methodology used has been documented in the scientific literature (Garcia & Diaz, JSCC, 27, (1976) 379-398). JMP™ analytical software was used to calculate the statistics (student's t-test at 95% confidence level). The percentage reduction in combing forces was calculated using the following Equation 5.

$$\% Reduction in Combing Forces = ( 1 - ( Combing Force of Treatment \text{} / Combing Force of Control ) ) \times 100 \qquad Equation 5$$

A statistical reduction in friction was seen for the 1× Method Treated group and the 3× Method Treated group compared to the Control group, with the 1× Method Treated showing a 91.92% reduction in wet combing force compared with the control for the European hair and showing a 92.15% reduction in wet combing force compared with the control for the Curly hair, and with the 3× Method Treated showing a 90.84% reduction in wet combing force compared with the control for the European hair and showing a 90.96% reduction in wet combing force compared with the control for the Curly hair. See Table 8. This demonstrates that the methods and compositions herein improve manageability and provide detangling benefits and lower combing friction.

TABLE 8

| Group | N | Mean | Std. Dev. | Std. Err. Mean |
|---|---|---|---|---|
| Control - | 8 | 150.29 | 7.13 | 2.52 |
| European | | | | |
| 1x Method | 8 | 12.14* | 0.47 | 0.17 |
| Treated - | | | | |
| European | | | | |
| 3x Method | 8 | 13.76* | 1.33 | 0.47 |
| Treated - | | | | |
| European | | | | |
| Control - Curly | 8 | 176.58 | 6.56 | 2.32 |
| 1x Method | 7 | 13.87* | 0.63 | 0.24 |
| Treated - Curly | | | | |
| 3x Method | 8 | 15.97* | 0.52 | 0.18 |
| Treated - Curly | | | | |

*statistically different from the Control

Split End Repair: Hair was tested to demonstrate the ability of the treatment method using the composition to close split ends of hair fibers. Imaging and grading of split-end fibers were performed.

A curly multi-ethnic brown tress (Walker type 3A) (Curly) and a European medium brown hair tress (European) were tested, 50 randomly picked hair fibers per tress treatment were evaluated.

The tresses were divided into two treatment groups, a 1× Method Treated group and 3× Method Treated group. The 1× Method Treated group tresses were 1) washed with a sodium laureth sulfate (SLES) solution containing 15% SLES, the solution being used at 10% by weight of the tresses for the shampoo, by applying the shampoo at 10% by weight of the tresses (0.1 ml/g of hair), massaged for 30 seconds, and then rinsed for 30 seconds, 2) the composition of Table 6 was applied to the damp hair at 10% by weight of the tresses (0.1 ml/g of hair), massaged for 30 seconds. The 3× Method Treated group followed the method for the 1× Method Treated group but before repeated twice steps 1) and 2) for a total of three composition application cycles before post treatment imaging.

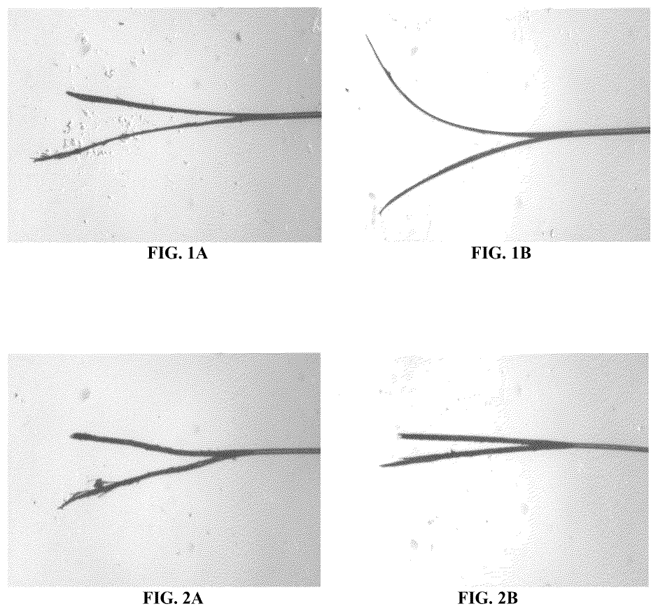

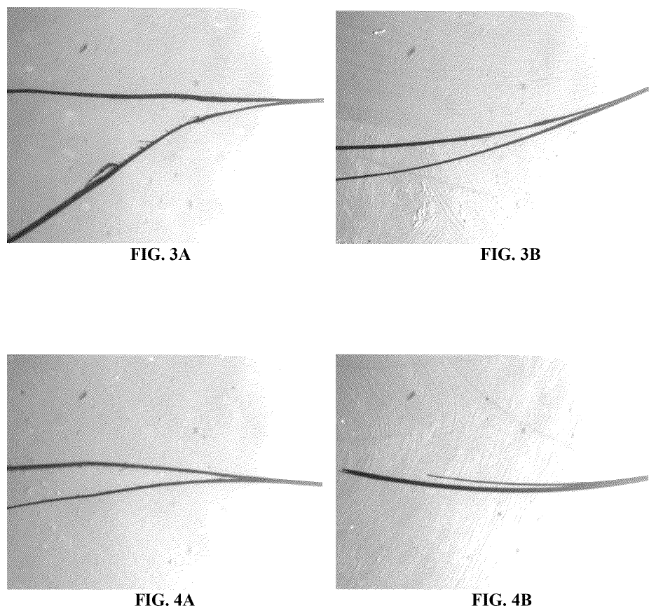

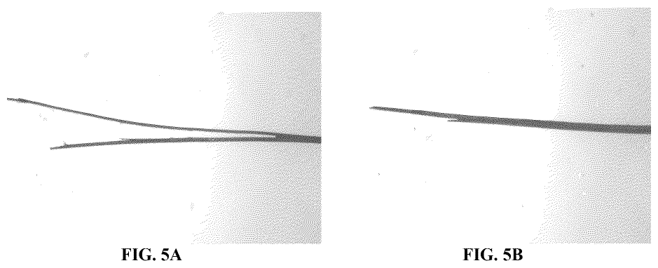

Determining whether the treatment will seal split ends was tested by comparing initial and post-treatment split end images. The procedure to test for closure of split ends followed the published procedure by Wright and Szerszen (Ending the Cycle of Split Ends, Cosmetics & Toiletries, November 2011, p 804), although this approach was introduced previously by Rigoletto et al. (Semi-permanent split end mending with a polyelectrolyte complex, J. Cosmet Sci., 58, 451-476, 2007; Polyelectrolyte complex for mending damaged hair, Cosmetics & Toiletries, 124 (3) 2009). Split ends were graded on a 5-point grading scale outlined in Table 9. Examples of the rating are shown FIGS. 1 to 5 where the split end of each fiber is imaged using a 25× stereo microscope before and after treatment.

TABLE 9

| Rate | Closure Description |
|---|---|
| 1 | No Closure |
| 2 | Minimal partial closure~less than 30% angle closure |
| 3 | Moderate degree of partial closure~between 30%-70% angle |
| | closure |
| 4 | Reasonable degree of partial closure~more than 70% angle closure |
| 5 | Complete closure |

The results show that the method for the 1× Method Treated group and the 3× Method Treated group were effective at closing split ends for both the European and Curly hair types. The data shows that at least 84% of split ends were at least partially closed and a near majority or majority of split ends are completely closed by both treatments. See Table 10.

TABLE 10

| | | | | Total at |
|---|---|---|---|---|
| | | | | Least |
| | | Partially | Completely | Partially |
| | | Closed | Closed | Closed |
| | | (only 3 and | (only 5 | (3, 4, and |
| | N | 4 Rating) | Rating) | 5 Rating) |
| Group | (fibers) | (%) | (%) | (%) |
| 1x Method Treated - | 50 | 20 | 64 | 84 |
| European | | | | |
| 3x Method Treated - | 50 | 20 | 64 | 86 |
| European | | | | |
| 1x Method Treated - | 50 | 42 | 46 | 88 |
| Curly | | | | |
| 3x Method Treated - | 50 | 36 | 50 | 86 |
| Curly | | | | |

Heat Protection: Hair was tested to demonstrate the ability of the treatment method using the composition to protect hair against heat damage. Heat damage was induced in this test using a flat iron at 232.2° C. (450° F.).

A curly multi-ethnic brown tress (Walker type 3A) (Curly) and a European medium brown hair tress (European) were tested, six hair fibers per treatment group were tested with High Pressure Differential Scanning calorimetry.

The tresses were divided into three treatment groups, a Method Treated group, a Heat Control group, and a No-Heat Control group. The Method Treated group tress was 1) washed with a sodium laureth sulfate (SLES) solution containing 15% SLES, the solution being used at 10% by weight of the tresses for the shampoo, by applying the shampoo at 10% by weight of the tresses (0.1 ml/g of hair), massaged for 30 seconds, rinsed for 30 seconds, then towel dried, 2) the composition of Table 6 was applied to the damp hair at 10% by weight of the tresses (0.1 ml/g of hair), and massaged for 30 seconds, and 3) the tress was blow-dried using medium-heat and high-velocity for 18 minutes and then flat ironed at 232.2° C. (450° F.) for ten strokes at a rate of ten seconds per stroke from root to tip. The treatment was repeated two additional times, for a total of three treatment cycles. Then the tress was 4) washed with a sodium laureth sulfate (SLES) solution containing 15% SLES, the solution being used at 10% by weight of the tresses for the shampoo, by applying the shampoo at 10% by weight of the tresses (0.1 ml/g of hair), massaged for 30 seconds, rinsed for 30 seconds and then 5) soaked while stirring overnight in four liters of deionized water and then air dried overnight. The Heat Control group tress was treated in the same way as the Method Treated group tress except step 2 did not apply the composition of Table 6, but instead used 1.0 ml of deionized water. The No-Heat Control group tress was treated in the same way as the Heat Control group except that step 3 was not performed.

Determining whether the treatment will protect from heat damage was tested by analyzing the middle section of the hair tress (not the root or the tip) by High Pressure Differential Scanning calorimetry (HP-DSC). Six samples were taken for each treatment. Denaturation temperature maximum (Td) of the tresses, that reflects relative thermal stability, was measured and change in enthalpic peak via endothermic peak area (ΔH) of the tresses, that reflects relative amount of crystalline, or ordered proteins, within a fiber, was measured. As has been reported (Milcazrek et al. The mechanisms and stability of thermal transitions in hair keratin, Colloid Polymer Science 270, 1106-1115 (1992)), keratin undergoes detectable transformations at various temperatures. These temperatures depend on treatments that the hair has been subjected, for example, bleaching decreases the transition peak temperature. According to published results, these transitions may be due to changes in keratin structure, cysteine bond arrangements, or strongly bound water in the keratin. HP-DSC can therefore provide insight into how a treatment may affect deep structural changes in the hair keratin. Analysis was performed on small hair samples (<10 mg). Peak temperatures as well as enthalpies are measured and reported. JMP™ analytical software was used to calculate the statistics (student's t-test at 95% confidence level). A one-way analysis of the data is performed on a distribution of Y (ΔH and Td) for each X (Treatment).

A statistical reduction in heat damage was seen for the Method Treated group compared to the Heat Control group for both Curly and European hair. When compared to the Heat Control group, the Method Treated group had a statistically significant increased denaturation temperature (Td), suggesting an increased matrix crosslink density for both Curly and European hair. See Table 11. Also, when compared to the Heat Control group, the Method Treated group had a statistically significant increased denaturation enthalpy (ΔH), suggesting an improved structural integrity of ordered hair proteins for both Curly and European hair. See Table 12.

TABLE 11

| | | Mean Td | Std. | Std. Err. |
|---|---|---|---|---|
| Group | N | (° C.) | Dev. | Mean |
| No-Heat Control - | 6 | 153.8\hat{ } | 1.0 | 0.4 |
| European | | | | |
| Heat Control - | 6 | 141.1* | 1.0 | 0.4 |
| European | | | | |
| Method Treated - | 6 | 145.8*\hat{ } | 1.1 | 0.4 |
| European | | | | |
| No-Heat Control - | 6 | 154.0\hat{ } | 0.5 | 0.2 |
| Curly | | | | |
| Heat Control - | 6 | 142.2* | 0.9 | 0.4 |
| Curly | | | | |
| Method Treated - | 6 | 145.7*\hat{ } | 0.6 | 0.3 |
| Curly | | | | |

*statistically different from the respective No-Heat Control
\hat{ }statistically different from the respective Heat Control

TABLE 12

| | | Mean Td | Std. | Std. Err. |
|---|---|---|---|---|
| Group | N | (° C.) | Dev. | Mean |
| No-Heat Control - | 6 | 16.8\hat{ } | 2.2 | 0.9 |
| European | | | | |
| Heat Control - | 6 | 10.5* | 0.7 | 0.3 |
| European | | | | |
| Method Treated - | 6 | 12.6*\hat{ } | 1.0 | 0.4 |
| European | | | | |
| No-Heat Control - | 6 | 17.5\hat{ } | 1.6 | 0.7 |
| Curly | | | | |
| Heat Control - | 6 | 11.4* | 1.4 | 0.6 |
| Curly | | | | |
| Method Treated - | 6 | 14.0*\hat{ } | 1.0 | 0.4 |
| Curly | | | | |

*statistically different from the respective No-Heat Control
\hat{ }statistically different from the respective Heat Control

Cuticle Sealing: Hair was tested to demonstrate the ability of the treatment method using the composition to reduce cracks in hair cuticles.

Curly multi-ethnic brown tresses (Walker type 3A), 9% bleached (Curly) and European Medium Brown tresses, 9% bleached were tested, three random hair fibers per treatment group were tested at three locations per fiber. All of the hair was first bleached three times by contacting the hair with a 9% hydrogen peroxide solution that has a pH of 10.2 and a temperature of 40° C. for 20 minutes at 40° C. with regular agitation and rinsed under running water between repeated bleaching and after the third bleaching. All of the hair was then fatigued for 3,000 cycles to induce damage to the hair.

The tresses were divided into three treatment groups, a 1× Method Treated group, a 3× Method Treated group, and a Control group. The 1× Method Treated group tresses were 1) dampened and the composition of Table 6 was applied to the damp hair at 10% by weight of the tresses (0.1 ml/g of hair), massaged for 30 seconds. The 3× Method Treated group followed the method for the 1× Method Treated group but repeated twice step 1) for a total of three composition application cycles with a wash between each composition application using a sodium laureth sulfate (SLES) solution containing 15% SLES, the solution being used at 10% by weight of the tresses for the shampoo, by applying the shampoo at 10% by weight of the tresses (0.1 ml/g of hair), massaged for 30 seconds, and then rinsed for 30 seconds. A Control group of tresses were not treated.

Determining whether the treatment will repair cracks in the cuticle of the hair was tested by imaging the hair by Scanning Electron Microscopy (SEM) on a JEOL JSM-IT200LV microscope. The hair fibers were attached to the surface of a 2.5 cm diameter stainless steel disc using double-sided copper tape and coated with Platinum to approximately 7 nm. The SEM pictures were recorded at 7 KV with a working distance of ˜11 mm at 600× and 2000× magnification. SEM is a surface analytical technique used to generate magnified topographic images of a material surface from 20× to over 100,000×. SEM uses a focused beam of high-energy electrons to generate a variety of signals at the surface of solid specimens. The field-emission cathode in the electron gun of an SEM provides narrower probing beams at low as well as high electron energy, resulting in both improved spatial resolution and minimized sample charging and damage. The signals that derive from electron-sample interaction reveal information about the sample, including external morphology (texture), crystalline structure, and orientation of materials making up the sample. SEM is often used to visualize the surface of hair under high magnification and picture the condition of protective cuticle scales. This technique can also provide information about the morphology of deposited surface coatings.

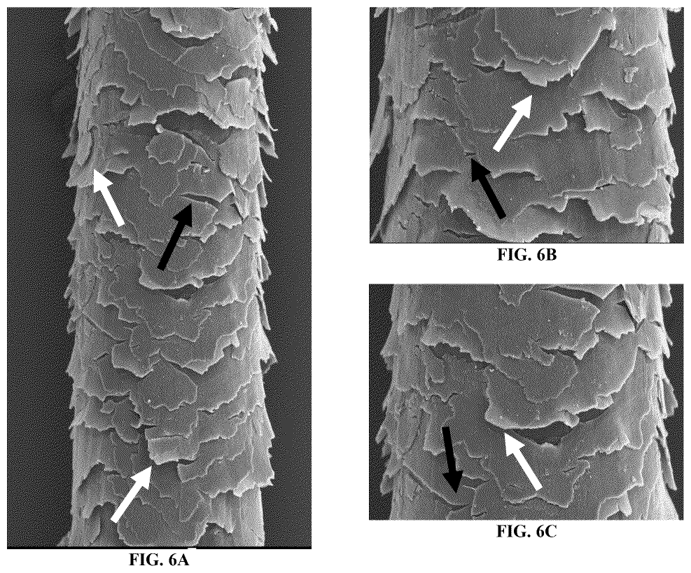

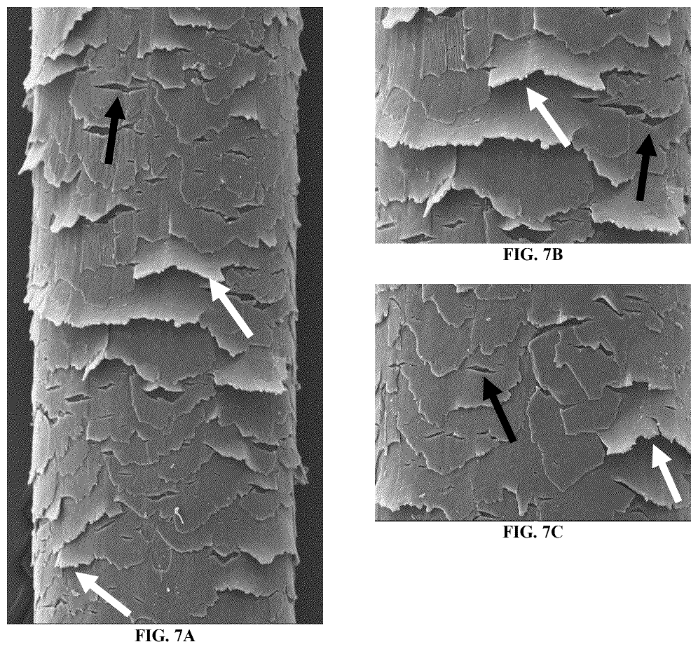

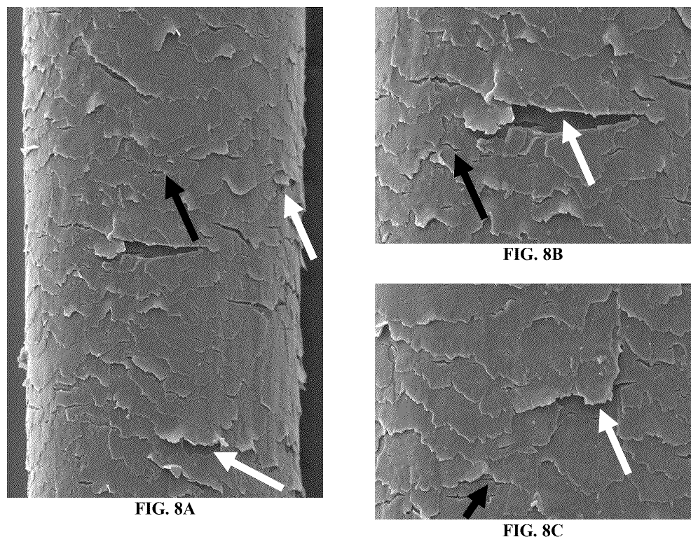

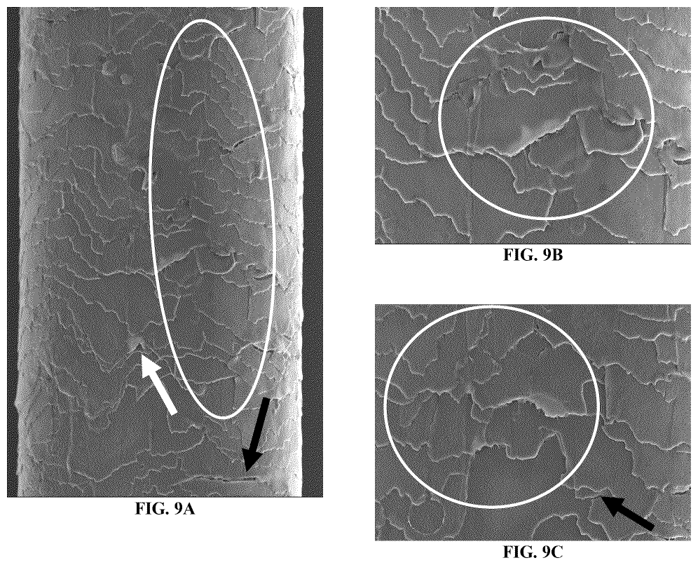

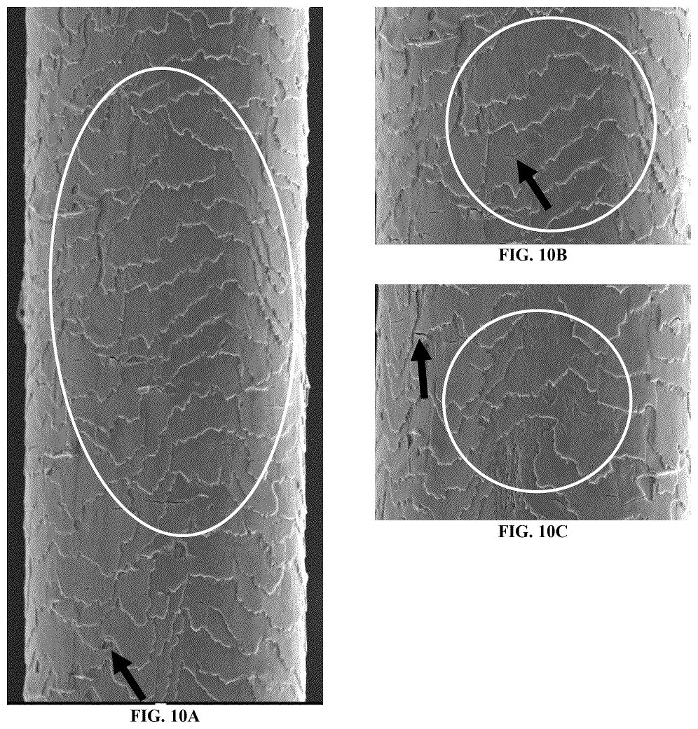

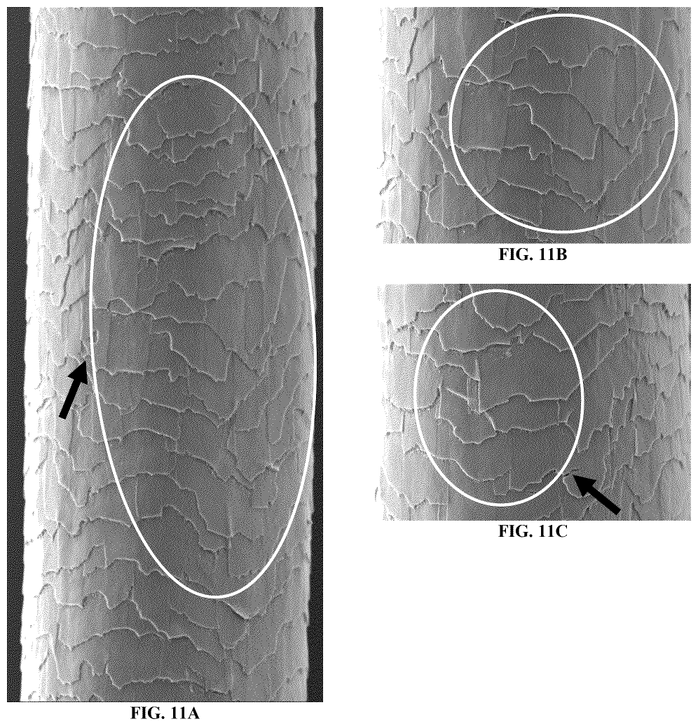

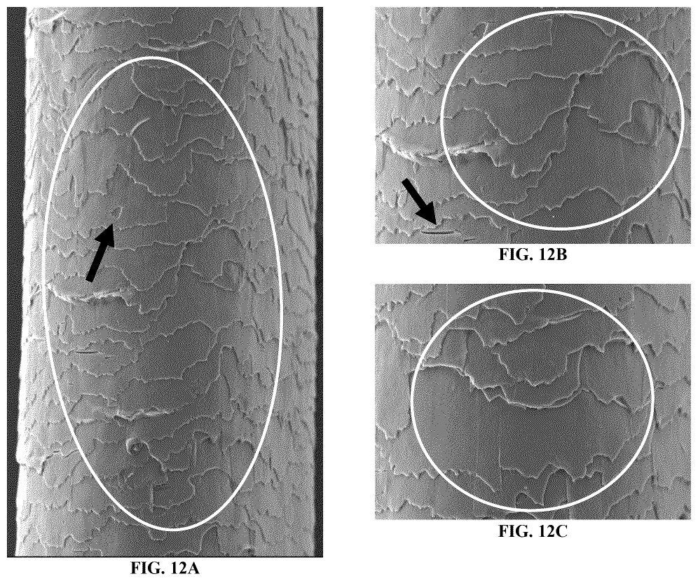

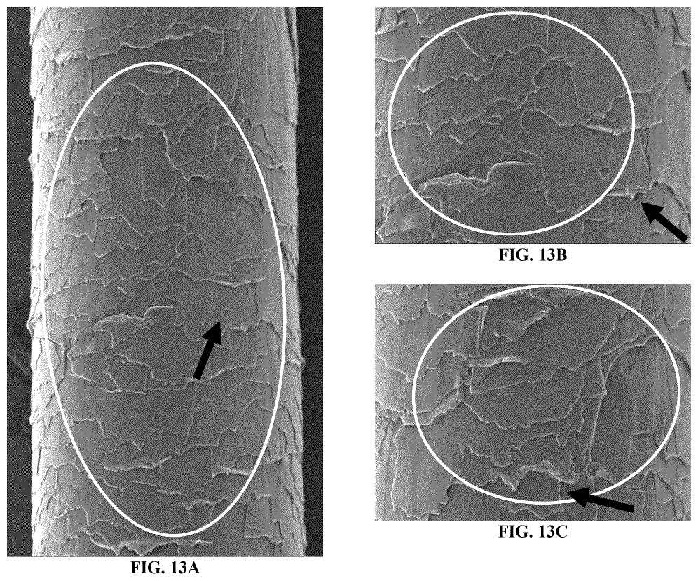

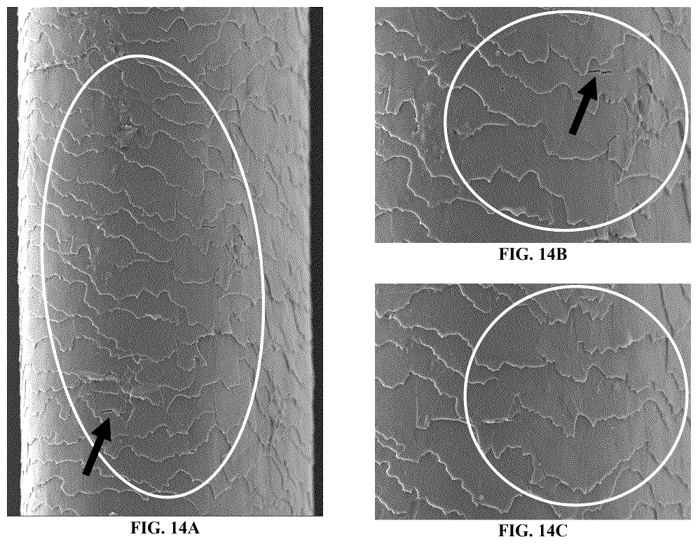

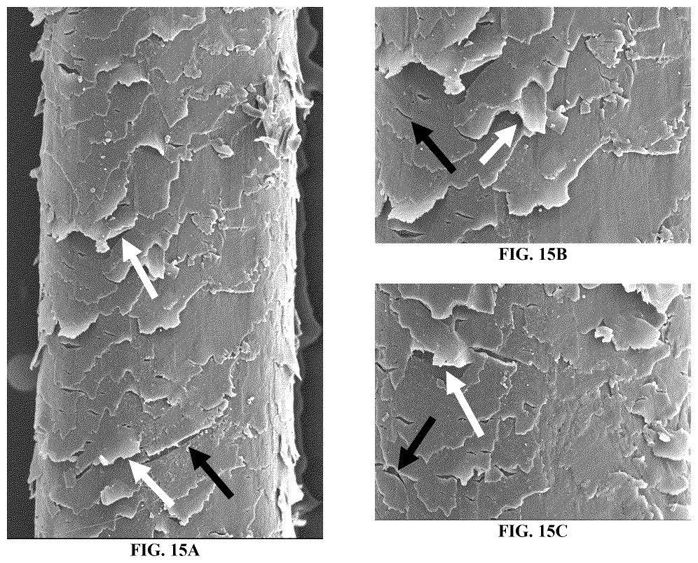

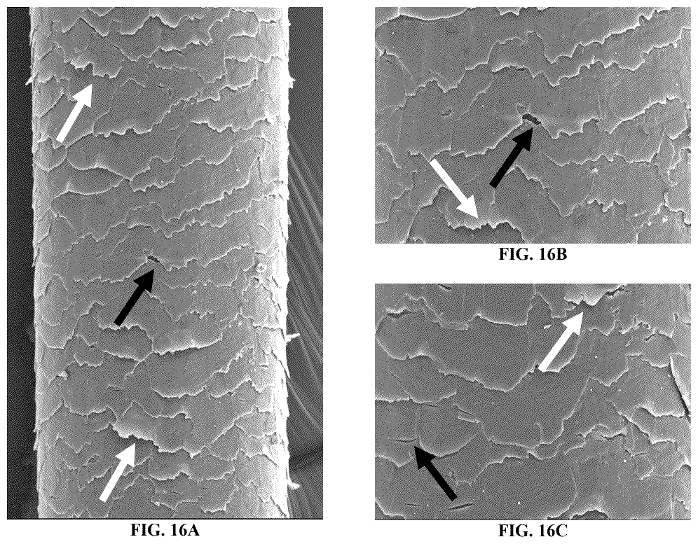

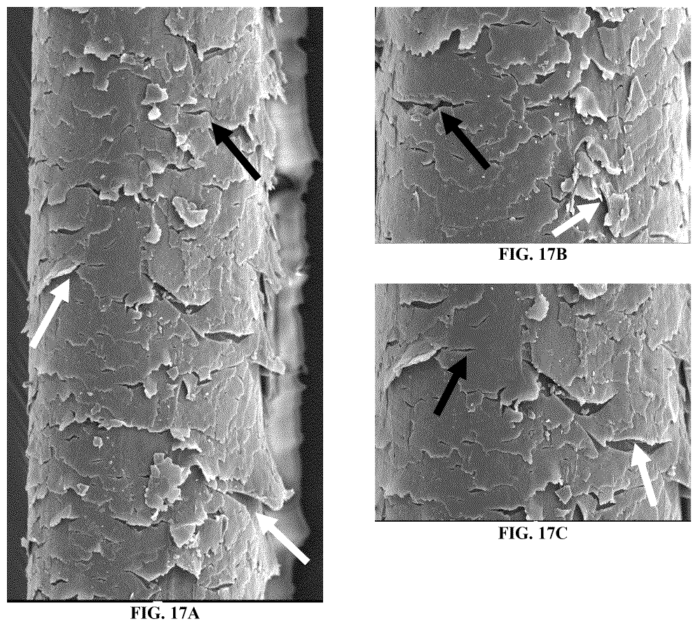

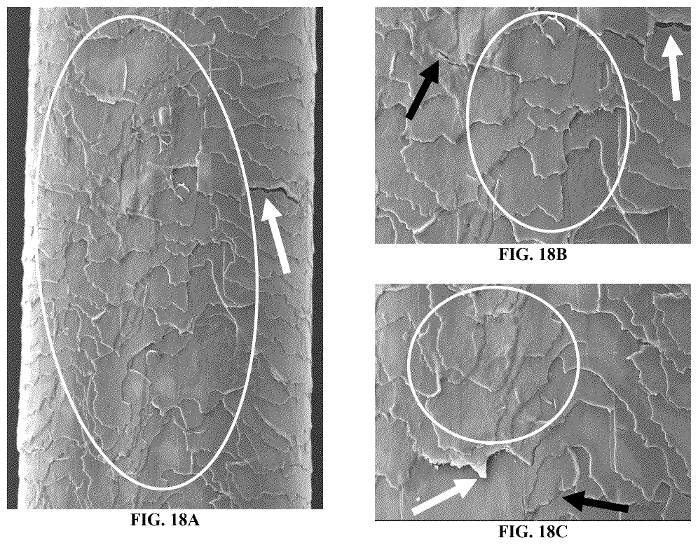

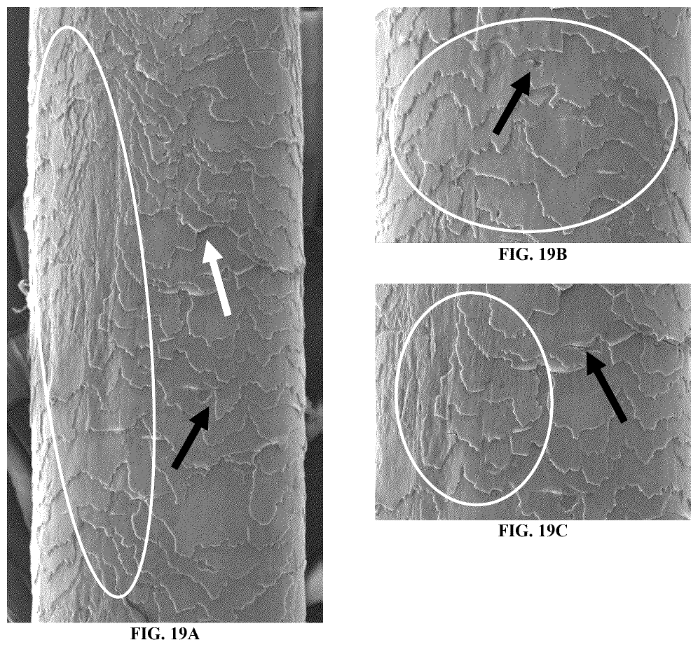

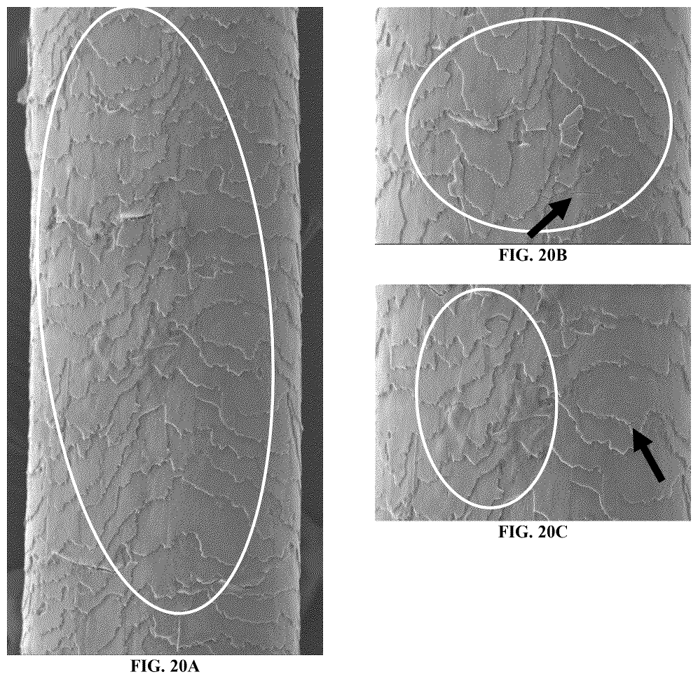

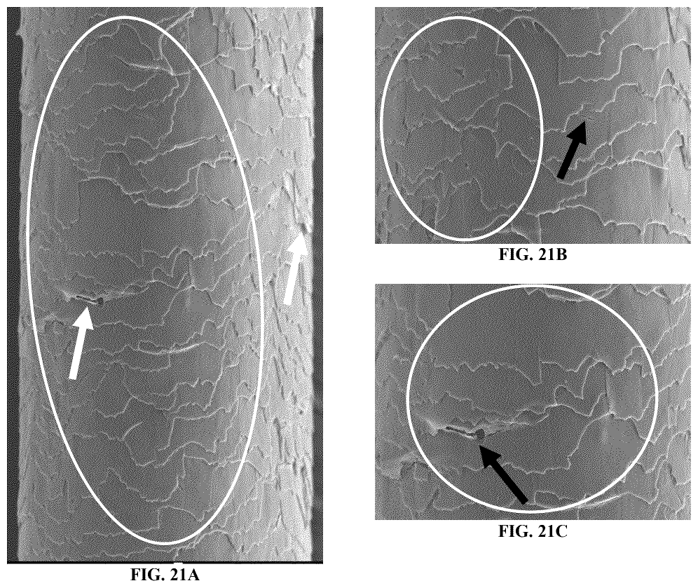

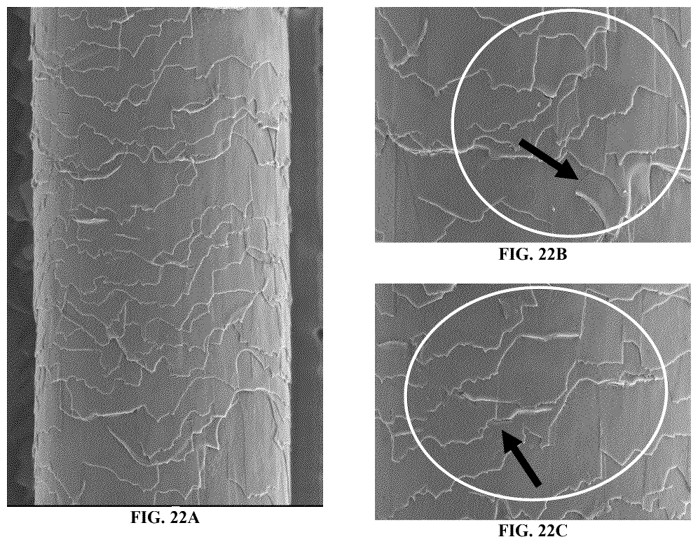

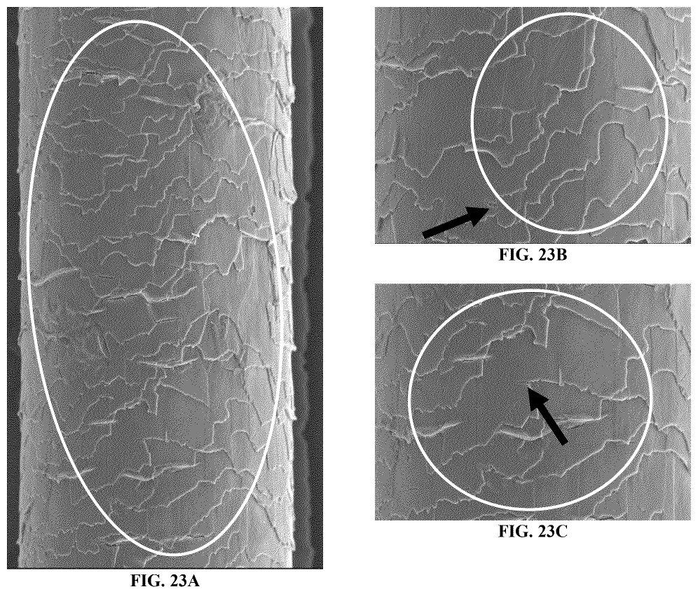

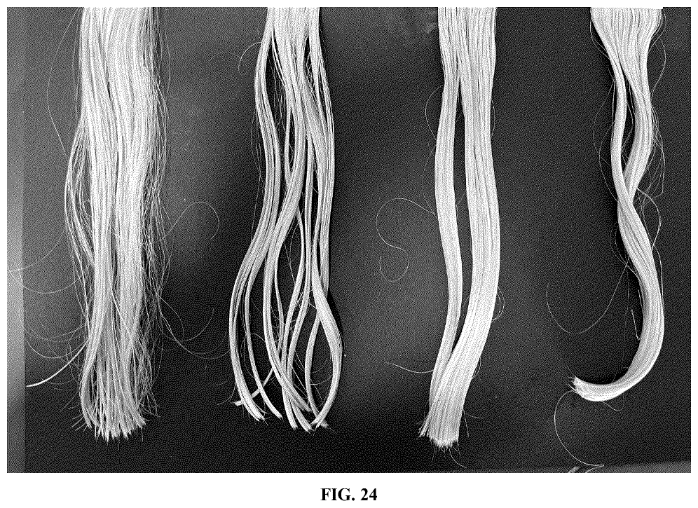

SEM image analysis shows that the 1× Method Treated group and the 3× Method Treated group is more smooth and has less lifted cuticle edges compared to the Control group for both hair types (Curly and European). See FIGS. 6 to 23. FIGS. 6 to 23 show cuticle lifting (some examples shown in white arrows) and cuticle cracks (some examples shown in black arrows). For the 1× Method Treated group and 3× Method Treated group it is possible to see lifted cuticle edges are smoothened (some examples shown circled in white) when compared to the Control group. For the 3× Method Treated group it is possible to see lifted cuticle edges are smoothened and a thin product layers is shown (circled in white) when compared to the Control group. Further, the 3× Method Treated group appears to have less cuticle lifting for both hair types compared to the 1× Method Treated group. This demonstrates that the methods and compositions herein improve the surface condition of damaged hair for both European and Curly hair.

### C. Example 3 (Comparison of Combination of Ingredients on Hair)

Wet Combing and Dry Combing Comparisons: Hair was tested to demonstrate the ability of the treatment method using the composition to smooth damaged hair via wet combing and via dry combing and to demonstrate the ability to reduce frizziness and fly aways after wet combing or dry combing.

3 g, 8″ long, dark-curl (Type 3) fine density tresses that had been bleached and then treated with 40 vol Developer were tested, one tress per group. The tresses were divided into four treatment groups: a Method Treated group; a Hydroxypropyl Guar Hydroxypropyltrimonium Chloride (HGHC) Treated group; a Commercial Hair Repairing Leave-In Conditioner (Commercial) group; and a Control group.

The Method Treated group tress was 1) washed with Olagoplex's No. 4C Clarifying Shampoo and then rinsed and 2) the composition of Table 6 was applied to the damp hair at 10% by weight of the tresses (0.1 ml/g of hair), massaged for 30 seconds, and rinsed for 30 seconds. The HGHC Treated group followed the method for the Method Treated group but the composition of Table 6 in step 2 was replaced with a composition similar to that of Table 6 except the composition did not contain any of Argania spinosa Kernel Oil, Algin, Sorbitan Oleate, Coco-Glucoside, Carrageenan, Xanthan Gum, and Chitosan and contained a smaller amount of Guar Hydroxypropyltrimonium Chloride, Caprylic/Capric Triglycerid, and Phenoxyethanol than the composition of Table 6 tested in the Method group but still within the range of concentrations of those respective ingredients (Guar Hydroxypropyltrimonium Chloride, Caprylic/Capric Triglycerid, and Phenoxyethanol) shown in Table 6. The Commercial Treated group followed the method for the Method Treated group but the composition of Table 6 in step 2 was replaced with a commercially available hair repairing leave-in conditioner from K18™, K18 BIOMIMETIC HAIRSCIENCE™ LEAVE-IN MOLECULAR REPAIR HAIR MASK™. A Control group of tresses were washed as described for the Method Treated procedure but the solution in step 2 was water. For the dry comb testing, the tresses were then blow-dried followed immediately by the dry combing measurements.

Determining whether the treatment will smooth the hair was tested by an Instron tensile tester to measure frictional forces while a hair tress (wet or dry) is pulled through a comb. A treatment that smooths hair can lower the frictional forces. Five replicates for each sample were performed. A similar methodology used has been documented in the scientific literature (Garcia & Diaz, JSCC, 27, (1976) 379-398). The percentage reduction in combing forces was calculated using the following Equation 5.

$$\% Reduction in Combing Forces = ( 1 - ( Combing Force of Treatment \text{} / Combing Force of Control ) ) \times 100 \qquad Equation 5$$

For the friction tests, a reduction in friction was seen for the HGHC Treated group, the Method Treated group, and the Commercial Treated group compared to the Control group, with the Method Treated group showing a greater reduction than the HGHC Treated group for both wet and dry combining and the Method Treated group showing a greater reduction than the Commercial Treated group for the dry combining. See Table 13. This demonstrates that the methods and compositions herein improve manageability and provide detangling benefits and provide lower combing friction than the commercial product tested for dry combing and similar combing friction to the commercial product tested for wet combing. Also, this demonstrates that the methods and compositions herein improve manageability and provide detangling benefits and provide lower combing friction than use of just Hydroxypropyl Guar Hydroxypropyltrimonium Chloride.

TABLE 13

| Group | Mean Max Force | % Change |
|---|---|---|
| Control - Wet Combing | 120.466 | n/a |
| HGHC Treated - Wet Combing | 35.945 | 70.16 |
| Method Treated - Wet Combing | 24.560 | 79.61 |
| Commercial Treated - Wet Combing | 20.276 | 83.17 |
| Control - Dry Combing | 571.777 | n/a |
| HGHC Treated - Dry Combing | 66.12 | 88.44 |
| Method Treated - Dry Combing | 53.208 | 90.69 |
| Commercial Treated - Dry Combing | 129.401 | 77.37 |

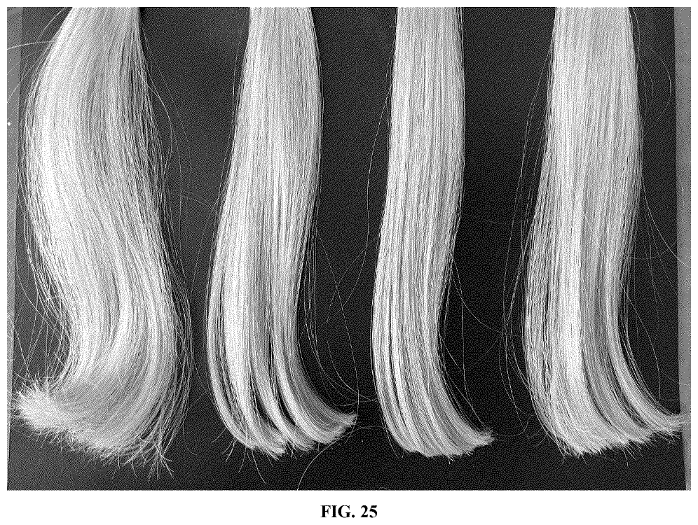

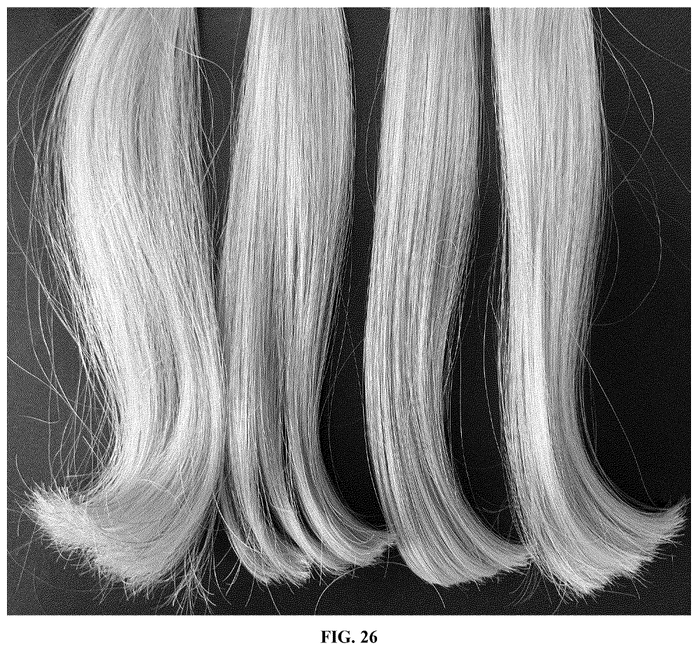

Determining whether the treatment will reduce frizziness and fly-aways was determined by visual inspection of the tresses immediately after the wet combing or dry combing tests. Images of the tresses were taken as shown in FIGS. 24 to 26. The order of tresses are from left to right, Control group, HGHC Treated group, Method Treated group, and Commercial Treated group. The results demonstrate that the Method Treated group shows a greater reduction in frizziness and fly aways than the HGHC Treated group for both wet and dry combining and the Method Treated group shows a reduction in frizziness and fly aways than the Commercial Treated group for both the wet combing and the dry combining. Also, all of the HGHC Treated group, the Method Treated group, and the Commercial Treated group show a greater reduction in frizziness and fly aways than the Control group. This demonstrates that the methods and compositions herein improve manageability and provide better reduction in frizziness and fly aways than the commercial product tested for both wet combing and dry combing. Also, this demonstrates that the methods and compositions herein improve manageability and provide better reduction in frizziness and fly aways when the composition contains all of Hydroxypropyl Guar Hydroxypropyltrimonium Chloride, Guar Hydroxypropyltrimonium Chloride, Argania Spinosa Kernel Oil, and Algin.

### D. Example 4 (Clinical Studies)

In a clinical study, it has been determined that the methods and composition disclosed herein can, among other things, reduce split ends and can help hair look repaired, healthier, and less damaged. Many of the benefits of use of the method and composition improved over the 14 day trial. Further, 74% of participants marked “Strongly Agree” or “Agree” when asked if they “like this product more than my current leave-in conditioner.” A summary of results are found in Tables 14 to 22 and the outline of the clinical study methods and results used to determine the properties of the ingredients are provided below.

After the first day of treatment, after 7 days of the trial, and at the end of a 14 day trial consumers were required to complete self-assessment surveys about the products' attributes. Seventy-four participants between the ages 18-45 completed the fully blinded study and the brand was not divulged. 14% of the participants had Type 1: Straight hair, 16% had Type 2A: Slight Wave, 18% had Type 2B: Loose Wave, 16% had Type 2C: Wavy, 11% had Type 3A: Loose Curl, 12% had Type 3B: Curly, 3% had Type 3C: Tight Curls, 5% had Type 4A: Loose Coils, and 5% had Type 4B: Coiled hair.

All participants hair were instructed to (1) shampoo and condition their hair with their regular products as normal, (2) apply the composition of Formula 6 to clean, damp hair, using 1 pump of the container of the composition for fine hair and using at least 2 pumps for medium to course hair and more if needed and on dry ends of hair between washes if needed, and (3) style the hair with stylers and style as normal. Participants were to follow these steps on the first day of the trial and then at least two to three times per week.

The results of the survey are shown in Tables 14 through 22.

TABLE 14

| | “Strongly Agree” and | | |
|---|---|---|---|
| | “Agree” (%) | | |
| Application and Product Attribute Ratings | Day 1 | Day 7 | Day 14 |
| Is easy to apply | 97 | 99 | 99 |
| Does not feel sticky | 97 | 95 | 99 |
| Is easy to distribute through the hair | 93 | 96 | 97 |
| Absorbs quickly into hair | 95 | 96 | 97 |
| The product does not flake | 93 | 96 | 97 |
| The product worked well with my other stylers | 91 | 95 | 96 |
| The formula texture is suitable for my hair type | 91 | 91 | 95 |
| Does not leave a visible residue on hair | 97 | 97 | 95 |
| Hair feels smooth | 95 | 95 | 95 |
| Hair feels soft | 91 | 92 | 93 |
| Makes brushing/combing through hair easier | 91 | 89 | 92 |
| Leaves hair more manageable | 88 | 92 | 92 |
| Hair has a healthy-looking shine | 82 | 84 | 92 |
| Does not make hair feel weighed down/heavy | 95 | 96 | 91 |
| Easy to detangle hair after using this product | 92 | 91 | 91 |
| Hair feels moisturized | 89 | 89 | 91 |
| The product can be layered with stylers without visible residue | 91 | 91 | 89 |
| Does not leave behind a greasy or oily residue | 95 | 86 | 89 |
| Provides weightless hydration | 88 | 91 | 89 |
| Hair looks healthier | 78 | 86 | 89 |
| Hair feels less damaged | 74 | 88 | 89 |
| Hair looks less damaged | 76 | 81 | 89 |
| Hair feels hydrated without leaving hair feeling heavy | 88 | 86 | 88 |
| My ends look smoother | 80 | 84 | 88 |
| Split ends are less visible | 68 | 81 | 86 |
| My ends look healthier | 74 | 84 | 86 |
| Hair appears frizz-free | 81 | 86 | 86 |
| Hair looks repaired | 69 | 77 | 86 |
| Hair feels healthier | 81 | 88 | 85 |
| Visibly reduces signs of hair damage | 68 | 73 | 85 |
| Hair feels deeply conditioned | 85 | 81 | 84 |
| Seals the appearance of split ends | 59 | 77 | 84 |
| Revives hair health | 66 | 78 | 82 |
| I was able to reapply on dry hair without weighing down strands | 57 | 80 | 81 |
| On day 1 after application, hair feels healthier instantly | 80 | N/A | N/A |
| On day 1 after application, hair instantly looks healthier | 73 | N/A | N/A |
| On day 1, my ends look immediately healthier after 1 use | 65 | N/A | N/A |
| On day 1, visibly reversed signs of hair damage after 1 use | 61 | N/A | N/A |
| On day 7, my hair feels healthier than it did a week ago | N/A | 81 | N/A |
| On day 7, my hair looks healthier than it did a week ago | N/A | 80 | N/A |
| On day 7, my hair looks healthier with every use | N/A | 80 | N/A |
| On day 7, my hair feels healthier with every use | N/A | 80 | N/A |
| On day 7, my ends look healthier with every use | N/A | 78 | N/A |
| On day 7, my ends feel healthier with every use | N/A | 76 | N/A |
| On day 14, my hair looks healthier than it did two weeks ago | N/A | N/A | 86 |
| On day 14, my hair looks healthier with every use | N/A | N/A | 86 |
| On day 14, my ends look healthier with every use | N/A | N/A | 86 |
| On day 14, my ends feel healthier with every use | N/A | N/A | 86 |
| On day 14, my hair feels healthier than it did two weeks ago | N/A | N/A | 85 |
| On day 14, my hair feels healthier with every use | N/A | N/A | 84 |
| On day 14, I like this product more than my current leave-in | N/A | N/A | 74 |
| conditioner | | | |

N/A = Not Applicable

TABLE 15

| | “Yes” % | | |
|---|---|---|---|
| | Day | Day | Day |
| Product Attribute Ratings (Yes or No Questions) | 1 | 7 | 14 |
| Do you like the texture & consistency of the | 99 | 96 | 99 |
| product? | | | |
| Do you like the smell of the product? | 82 | 82 | 81 |

TABLE 16

| In the past 7 days, how many times did you use the | Participants (%) | |
|---|---|---|
| product? | Day 7 | Day 14 |
| 1 | 0 | 3 |
| 2 | 9 | 5 |
| 3 | 30 | 23 |
| 4 | 34 | 31 |
| 5 | 12 | 14 |
| 6 | 7 | 7 |
| 7 | 8 | 18 |

TABLE 17

| | Participants |
|---|---|
| On a scale from 1 to 10, how effective was this | at Day 14 |
| product at addressing your concerns? | (%) |
| 10 - Very Effective | 31 |
| 9 | 23 |
| 8 | 26 |
| 7 | 8 |
| 6 | 7 |
| 5 | 1 |
| 4 | 0 |
| 3 | 4 |
| 2 | 0 |
| 1 - Ineffective | 0 |
| Average Effectiveness | 8.4 |

TABLE 18

| | Participants |
|---|---|
| On a scale from 1 to 10, how happy were you with | at Day 14 |
| the results from this product? | (%) |
| 10 - Very Happy | 46 |
| 9 | 18 |
| 8 | 15 |
| 7 | 8 |
| 6 | 5 |
| 5 | 4 |
| 4 | 0 |
| 3 | 4 |
| 2 | 0 |
| 1 - Unhappy | 0 |
| Average Happiness | 8.6 |

TABLE 19

| | Participants |
|---|---|
| How likely would you be to recommend this product | at Day 14 |
| to a friend/family member? | (%) |
| 5 - I would recommend | 65 |
| 4 - Likely | 24 |
| 3 - Not sure | 4 |
| 2 - Unlikely | 5 |
| 1 - I would not recommend | 1 |
| Average Recommendation Intent | 4.5 |

TABLE 20

| | Participants |
|---|---|
| | at Day 14 |
| How likely would you be to purchase this product? | (%) |
| 5 - I would purchase | 61 |
| 4 - Likely | 22 |
| 3 - Not sure | 8 |
| 2 - Unlikely | 7 |
| 1 - I would not purchase | 3 |
| Average Purchase Intent | 4.3 |

TABLE 21

| | Participants |
|---|---|
| How likely would you be to continue to use this | at Day 14 |
| product? | (%) |
| 5 - I would continue to use | 70 |
| 4 - Likely | 12 |
| 3 - Not sure | 11 |
| 2 - Unlikely | 7 |
| 1 - I would not continue to use | 0 |
| Average Purchase Intent | 4.5 |

TABLE 22

| On a scale from 1 to 5, with 1 being the lowest and 5 | Participants |
|---|---|
| being the highest, how many stars would you give | at Day 14 |
| this product? | (%) |
| 5 stars | 49 |
| 4 stars | 36 |
| 3 stars | 9 |
| 2 stars | 5 |
| 1 star | 0 |
| Average Stars | 4.3 |

1. A method of treating a person's hair, the method comprising applying to the person's hair a composition comprising:
(a) hydroxypropyl guar hydroxypropyltrimonium chloride;
(b) Argania spinosa kernel oil;
(c) guar hydroxypropyltrimonium chloride; and
(d) algin,
wherein application of the composition to the hair (1) seals at least 80% of split ends in the hair, (2) seals cracks in hair cuticles, (3) protects the hair from damage from heat up to 205° C., (4) reduces hair frizziness, and/or (5) reduces hair fly-aways.

2. The method of claim 1, wherein the hair comprises split ends and wherein application of the composition to the hair seals greater than 90% of split ends.

3. The method of claim 1, wherein the hair comprises cracks in the hair cuticles and wherein application of the composition to the hair seals at least a portion of the cracks in the hair cuticles.

4. The method of claim 1, wherein application of the composition to the hair reduces hair frizziness and/or reduces hair fly-aways.

5. The method of claim 1, further comprising heat treating the hair and wherein application of the composition to the hair at least partially protects the hair from damage from heat up to 205° C.

6. The method of claim 1, wherein application of the composition to the hair increases the elongation at break of individual hair fibers in the hair.

7. The method of claim 1, wherein application of the composition to the hair increases the tensile strength of the hair.

8. The method of claim 1, wherein the composition is applied to wet hair.

9. The method of claim 1, wherein the composition is not rinsed from the hair within 18 hours.

10. The method of claim 1, wherein the composition includes less than 0.5 wt. % of a silicone.

11. The method of claim 1, wherein the composition has:
a viscosity of 50,000-150,000 cps;
a specific gravity of 0.96 to 0.99; and
a pH of 4.0 to 5.0.

12. The method of claim 1, wherein the composition comprises, by weight of the composition:
0.25 to 0.75 wt. % of hydroxypropyl guar hydroxypropyltrimonium chloride;
0.5 to 0.5 wt. % Argania spinosa kernel oil;
0.1 to 0.5 wt. % guar hydroxypropyltrimonium chloride; and
0.001 to 0.2 wt. % algin.

13. The method of claim 1, wherein the composition further comprises bis-aminopropyl diglycol dimaleate.

14. The method of claim 13, wherein the composition comprises 0.001 to 0.2 wt. % bis-aminopropyl diglycol dimaleate by weight of the composition.

15. The method of claim 13, wherein the bis-aminopropyl diglycol dimaleate forms at least two disulfide bonds with one or more broken disulfide bond of the hair.

16. The method of claim 1, wherein the composition further comprises:
a fructooligosaccharide;
Beta vulgaris root extract;
arginine;
aspartic acid;
glycine;
alanine;
serine;
valine;
proline;
threonine;
isoleucine;
histidine; and/or
phenylalanine.

17. The method of claim 16, wherein the composition comprises, by weight of the composition:
0.1 to 2 wt. % of the fructooligosaccharide;
0.1 to 1.5 wt. % of Beta vulgaris root extract;
0.01 to 1 wt. % of arginine;
0.01 to 0.5 wt. % of aspartic acid;
0.001 to 0.1 wt. % of glycine;
0.001 to 0.1 wt. % of alanine;
0.001 to 0.1 wt. % of serine;
0.001 to 0.1 wt. % of valine;
0.0001 to 0.1 wt. % of proline;
0.0001 to 0.1 wt. % of threonine;
0.0001 to 0.1 wt. % of isoleucine;
0.0001 to 0.1 wt. % of histidine; and/or
0.0001 to 0.1 wt. % of phenylalanine.

18. The method of claim 1, wherein the composition further comprises carrageenan, xanthan gum, chitosan, sodium benzoate, caprylic/capric triglyceride, sorbitan oleate, coco-glucoside, potassium lactate, lactic acid, sodium pyrrolidone carboxylic acid, pyrrolidone carboxylic acid, sodium lactate, and/or phenoxyethanol.

19. The method of claim 18, wherein the composition comprises, by weight of the composition:
0.1 to 2 wt. % phenoxyethanol;
0.1 to 1 wt. % of caprylic/capric triglyceride;
0.1 to 1 wt. % of sodium lactate;
0.01 to 1 wt. % of potassium lactate;
0.01 to 1 wt. % of sodium pyrrolidone carboxylic acid;
0.01 to 0.5 wt. % of pyrrolidone carboxylic acid;
0.001 to 0.5 wt. % of lactic acid;
0.001 to 0.1 wt. % carrageenan;
0.001 to 0.1 wt. % xanthan gum;
0.001 to 0.1 wt. % chitosan;
0.001 to 0.2 wt. % sorbitan oleate;
0.001 to 0.2 wt. % coco-glucoside; and/or
0.00001 to 0.01 wt. % sodium benzoate.

20. The method of claim 1, wherein the composition is a leave in conditioner.
